(19) United States
(12) Patent Application Publication
(10) Pub. No.: US 2024/0391903 A1
(43) Pub. Date: Nov. 28, 2024

(54) AUTOTAXIN-INHIBITORS

(71) Applicant: UNIVERSITAET MUENSTER, Muenster (DE)

(72) Inventors: Robert NITSCH, Muenster (DE); Udo DANNLOWSKI,, Muenster (DE); Florian KLOSS, Jena (DE); Johannes VOGT, Koeln (DE); Hector Manuel TORRES-GOMEZ, Jena (DE)

(73) Assignee: UNIVERSITAET MUENSTER, Muenster (DE)

(21) Appl. No.: 18/693,887

(22) PCT Filed: Sep. 21, 2022
(86) PCT No.: PCT/EP2022/076180

§ 371 Date: Mar. 20, 2024

(30) Foreign Application Priority Data
Sep. 21, 2021 (EP) 21197914.1

(51) Int. Cl. C07D 403/12; A61K 31/496

(52) U.S. Cl. CPC C07D 403/12; A61K 31/496

(57) ABSTRACT

The present invention relates to an Autotaxin (ATX) inhibitor according to the general formula (I), a pharmaceutical composition comprising said formula (I) and at least one excipient, their use in medicine as well as a process comprising converting compound (II) into compound (III).

### TECHNICAL FIELD OF THE INVENTION

The present invention relates to an Autotaxin (ATX) inhibitor according to the general formula (I), a pharmaceutical composition comprising said formula (I) and at least one excipient, as well as a process comprising converting the compound according to formula (II) into the compound according to formula (III).

### BACKGROUND ART

The brain, i.e. the central nervous system, is by far the most complex organ of the human body. Due to its complexity the brain is susceptible to a variety of illnesses, such as psychiatric, neurological and neurodegenerative diseases. These diseases include, but are not limited to, schizophrenia, depression, anxiety disorders, susceptibility to stress, panic disorders, bipolar disorder, Attention Deficit Hyperactivity Disorder (ADHD), eating disorders, but also multiple sclerosis, epilepsy, Alzheimer's disease and ischemic stroke.

As the functioning of the brain still today is only poorly understood, the treatment of its illnesses still presents a significant unmet medical need.

In particular schizophrenia, depression and bipolar disorder are serious mental illnesses affecting about one in every hundred people in the western world and causes significant personal and familial suffering, as well as incurring societal and economic costs. The neurobiological causes of such illnesses can be traced to dysfunctions in synaptic transmission in the brain, and antipsychotics, which primarily rely on altering dopamine or serotonin signaling pathways, are currently recommended for treatment. However, these therapies cause frequent and sometimes serious side effects.

Recently, bioactive lipid signaling and protein-lipid interactions have been found to be involved in all steps of synaptic signaling processes. A genetic controller for this pathway, the phosphatase-like molecule “plasticity related gene 1” (PRG-1), which when “deactivated” causes behavioral deficits in mice that are indicative of mental illness, has been identified (Trimbuch et al., 2009). The genetic sequence of PRG-1 is highly conserved from laboratory animals to humans.

It has been disclosed in WO2017071799A1 that the recently reported genetic mutation (single nucleotide polymorphism (SNP)) R345T in PRG-1 interferes with the pathway through a loss of control over LPA synaptic levels and a subsequent increase in glutamate release at the synapse. This genetic mutation has a frequency of about 0.6%, corresponding to approximately 3.5 million European and 1.5 million US citizens. Further, in WO2017071799A1 a human-population cohort has been assessed using sensory and EEG measurements, and found that carriers of this genetic mutation exhibited reduced sensory gating—the ability to filter out irrelevant sensory information. Reduced sensory gating is associated with schizophrenia and other behavioral disorders.

Neurons in the human brain can be broadly divided into two classes: excitatory or inhibitory, depending on whether they tend to induce or suppress the generation of an action potential. Maintaining the correct balance of excitation and inhibition (E/I balance) ensures that neural activity is homeostatically regulated and stays in a narrow and safe range. Excitatory neurons are characterized by the release of the neurotransmitter glutamate and increased levels at the synapse lead to imbalances in the E/I systems. Dysfunctions related to this homeostatic system have been suggested to contribute to the pathophysiology of mental disorders (Harrison and Weinberger, 2005; Javitt et al., 2008). PRG-1 tightly controls the presence of lysophosphatidic acid (LPA), which in turn controls glutamate release at the synapse (Trimbuch et al. 2009).

Reinstating a normal E/I balance could provide a potential treatment for a range of mental and neurological illnesses.

In this newly identified pathway, as disclosed in WO2017071799A1, it has been found that the unregulated synthesis of LPA or uncontrolled synaptic LPA-levels lead to excessive glutamate release at the synapse and to a shift in the E/I balance. Therefore, reducing the lysophosphatidic acid (LPA) level in the brain may treat or prevent many central nervous system disorders.

It has been found and disclosed in WO2017071799A1, that deactivated PRG-1 leads to uncontrolled, i.e. excessive LPA levels. Therefore, it is promising to treat central nervous system disorders by activating or upregulating PRG-1.

LPA is synthesized by the protein autotaxin (ATX) which acts upstream of the LPA-LPA2/PRG-1 axis (Moolenaar & Perrakis, 2011). WO2017071799A1 discloses that the inhibition of autotaxin diminishes LPA levels.

Moreover, it has been shown in WO2017071799A1 that mice, which have an altered excitation/inhibition balance, known to be an endophenotype of psychiatric disorders related to schizophrenia in man (e.g. Harrison and Weinberger, 2005; Javitt et al., 2008), after treatment with an autotaxin inhibitor showed full recovery from altered prepulse inhibition (PPI), the best established mouse-phenotype related to psychiatric disorders such as schizophrenia (Davis, 1984; Swerdlow et al., 1994; Braff et al., 2001). That test assay is also used similarly in humans and assesses the capacity of the brain for sensory gating, found to be altered in individuals carrying a loss-of-function mutation in the PRG-1 gene, thus strongly indicating that autotaxin inhibitors may also be efficacious in humans.

It has been demonstrated in WO2017071799A1 in a mouse model that autotaxin inhibitors effectively reduce food intake after food deprivation, indicating that they may be used to treat eating disorders, or disorders that benefit from reduced food intake, such as obesity.

There is also evidence that suggests that this pathway plays a role in other psychiatric disorders, such as anxiety disorders and ADHD as well as in neurological disorders such as multiple sclerosis and ischemic stroke. For the latter, in WO2017071799A1 first data has been collected in animal models showing that infarct size and stroke-related motor deficits are significantly reduced by use of the ATX-inhibiting agent PF8380.

E/I balance has been suggested to be involved in a variety of psychiatric disorders, including schizophrenia and bipolar disorder, but also panic disorders, ADHD, and more generally in resilience—the body's innate resistance to stress and adversity. This therapeutic strategy may not only be suitable for the treatment of patients suffering from mental illness, but may be also used preventively in individuals that are particularly susceptible to mental health problems.

It is also thought that LPA regulation and via this pathway regulation of neuronal hyperexcitability plays an important role in many neurological conditions such as epilepsy, multiple sclerosis, Alzheimer's disease as well as stroke. For stroke, in WO2017071799A1 data has been disclosed that infarct size and stroke-related motor deficits are significantly reduced in a mouse model when PF8380 is administered.

Further, US 2012/0202827 A1 discloses that autotaxin inhibitors are suitable for the treatment of various cancer forms.

Thus, there is opportunity for several medical applications for further autotaxin inhibitors, which ideally show better pharmacodynamic and/or pharmacokinetic properties than the autotaxin inhibitors of the prior art.

### SUMMARY OF THE INVENTION

The invention relates to a compound according to general structure (I)

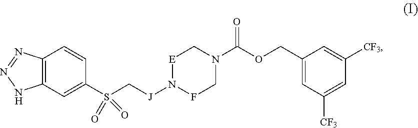

wherein
E is CH₂, C═O or CH(C₁-C₅)alkyl; preferably CH₂;
F is CH₂, C═O or CH(C₁-C₅)alkyl; preferably CH₂;
J is CH₂ or C═O, preferably CH₂;
or a pharmaceutically acceptable salt, solvate, enantiomer or hydrate thereof.

In a further aspect, the invention relates to a pharmaceutical composition comprising the compound according to the general structure (I) and at least one pharmaceutically acceptable excipient.

In a further aspect, the invention relates to the use of the compound according to general structure (I) for use in medicine.

In a further aspect, the invention relates to the compound of formula (I) or to the pharmaceutical composition for use in the prevention or in the treatment of diseases in a subject, in which the inhibition, regulation and/or modulation of autotaxin plays a role, preferably comprising reducing reducing the level of lysophosphatidic acid (LPA) in the targeted tissue of said subject, more preferably in the brain of said subject.

In a further aspect, the invention relates to the compound of formula (I) or the pharmaceutical composition for use in the prevention or in the treatment of a central nervous system disorder in a subject, comprising reducing the level of lysophosphatidic acid (LPA) in the brain of said subject or in the prevention or treatment of cancer.

In a further aspect, the invention relates to a process, preferably for the preparation of (III) comprising

a) the step of converting compound (II) into compound (III)

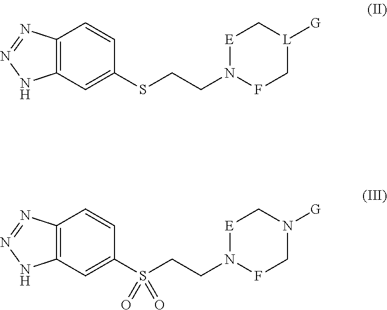

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl; and/or
   b) formation of compound (IV)

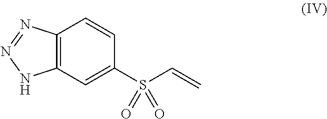

and/or
c) formation of compound (IV) and addition of compound (V) at the vinyl group of compound (IV).

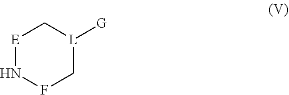

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆) alkyl, or —C(O)O(CH₂)ₙ(C₅-C₆)aryl; preferably —C(O)O(CH₂)ₙ(C₅-C₆)aryl;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

The compounds of the present invention showed improved pharmacokinetic properties as compounds in the prior art, while the autotaxin inhibition activity is maintained.

### BRIEF DESCRIPTION OF THE FIGURES

FIG. 1: MJK2134025 (compound (A)) shows comparable ATX-inhibtion as PF8380.

FIG. 2: Analysis of metabolism of MJK2134025 (compound (A)) in comparison to Diclofenac (as internal standard) and PF8380 using human liver microsomes (Corning) shows favorable decay time course.

FIG. 3: Microsomal stability assay comparing in vitro pharmacokinetic properties of MJK2134025 with prior art ATX lead inhibitors MSC2285264 and GLPG1690.

FIG. 4: Metabolic route of MJK2134025 (compound (A)) as assessed in human liver microsomes.

FIG. 5: Comparison between docking scores and binding energies.

FIG. 6: Docking scores and binding energies for the best poses selected by docking score (A) and binding energy (B).

FIG. 7: Distribution of binding energies for the studied compounds.

FIG. 8: C1-PC2 score plot from interaction energies calculated during docking for all the poses (A), for the best poses selected by docking (B).

FIG. 9: PC1-PC2 score plot from interaction fingerprints obtained after docking simulation for the best poses selected by docking.

FIG. 10: Best docking poses for the co-crystalized inhibitor PF-8380 (re-docking; A), MJK2134025 (B), 1 (C), 2 (D, MJK2234002), 3 (E), 4 (F), and 5 (G). A-G refer to structures in FIG. 12. Compound numbers 1-5 refer to FIGS. 5-9.

FIG. 11: Comparison of the binding pose of some compounds in place with the protein surface. PF-8380, 3, 1, 2.

FIG. 12: Summarized metabolization risk by CYP2C9 of PF-8380 (A), MJK2134025 (compound (A) of table 1) (B), and compounds 1 (C), 2 (D, MJK2234002), 3 (E), 4 (F), and 5 (G). Circles represent intrinsic reactivity and overall score; bigger size for higher score and darker colors for higher reactivity. Lines indicate Fe-accessibility.

FIGS. 13 bis 17: ATX-inhibitory activity of compounds MJK2234001, MJK2234002 and MJK2134025 as well as of comparison examples.

FIG. 18: Microsomal stability was determined for MJK2134025, MJK2234001, MJK2234002 and comparison compounds.

FIG. 19: MJK2134025 and GLPG 1690 were each administered as wet milled aqueous micro-suspensions at a concentration of 3 mg/g in 1% carboxymethyl cellulose and 0.5% Tween 80. Dosing of the formulations was performed at 10.0 g/kg body weight, corresponding to 30 mg/kg body weight. The food intake was measured.

FIG. 20: The plasma concentration of MJK2134025 and GLPG 1690 at 30 mg/kg oral dose measured over time.

FIG. 21 displays the results of example 5.7.

### DETAILED DESCRIPTION OF THE INVENTION

The solution of the present invention is described in the following, exemplified in the appended examples, illustrated in the Figures and reflected in the claims.

### Definitions

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

The term “alkyl” refers to a monoradical of a saturated straight or branched hydrocarbon. Preferably, the alkyl group comprises from 1 to 5 carbon atoms, i.e., 1, 2, 3, 4, 5 carbon atoms, more preferably 1 to 3 carbon atoms, most preferably 1 carbon atom. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, and the like.

The term “aryl” refers to a monoradical of an aromatic cyclic hydrocarbon. Preferably, the aryl group contains 5 to 6 carbon atoms which is arranged in one ring (e.g., phenyl). Exemplary aryl groups include cyclopropenylium, cyclopentadienyl, phenyl. Preferably, “aryl” refers to a monocyclic ring containing 6 carbon atoms or an aromatic bicyclic ring system containing 10 carbon atoms. Preferred examples are phenyl and naphthyl.

The term “halogen” means fluoro, chloro, bromo, or iodo.

A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, a hydroxy acid, such as citric acid, lactic acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate wherein the solvent is water.

“Enantiomers” are a pair of stereoisomers which are non-superimposable mirror-images of each other.

### Compounds of the Invention

The invention relates to a compound according to general structure (I)

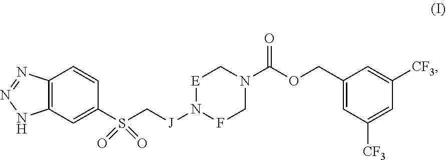

wherein
E is CH₂, C═O or CH(C₁-C₅)alkyl; preferably CH₂;
F is CH₂, C═O or CH(C₁-C₅)alkyl; preferably CH₂;
J is CH₂, or C═O, preferably CH₂
or a pharmaceutically acceptable salt, solvate, enantiomer or hydrate thereof.

An example wherein E and F is CH₂, is compound (A) in table 1.

An example wherein E or F is C═O, is compound (B) in table 1.

An example wherein E and/or F are CH(C₁-C₅)alkyl, is compound (C) and (D) in table 1.

An example wherein J is C═O is compound (E) in table 1.

Preferably, if J is C═O, E and F are not C═O.

TABLE 1
Examplary compounds of the present invention

| | |
|---|---|
| 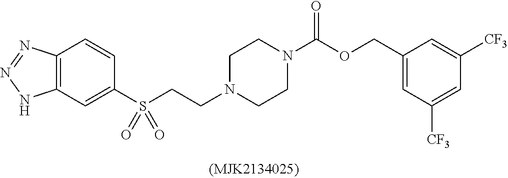 [SMILES: O=C(OCc1cc(C(F)(F)F)cc(C(F)(F)F)c1)N1CCN(CCS(=O)(=O)c2ccc3nn[nH]c3c2)CC1] | (A) |
| 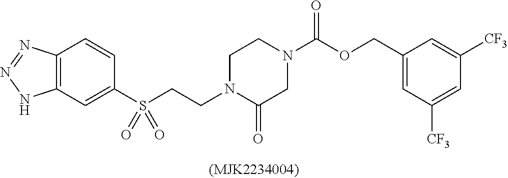 [SMILES: O=C1CN(C(=O)OCc2cc(C(F)(F)F)cc(C(F)(F)F)c2)CCN1CCS(=O)(=O)c1ccc2nn[nH]c2c1] | (B) |
| 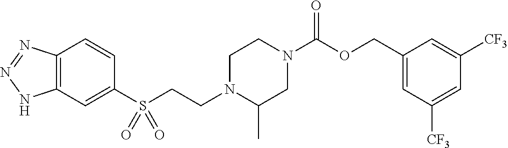 [SMILES: CC1CN(C(=O)OCc2cc(C(F)(F)F)cc(C(F)(F)F)c2)CCN1CCS(=O)(=O)c1ccc2nn[nH]c2c1] | (C) |
| 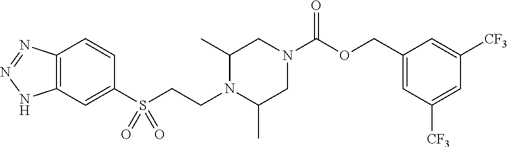 [SMILES: CC1CN(C(=O)OCc2cc(C(F)(F)F)cc(C(F)(F)F)c2)CC(C)N1CCS(=O)(=O)c1ccc2nn[nH]c2c1] | (D) |
| 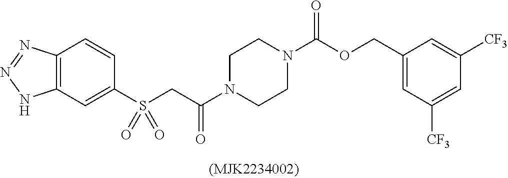 [SMILES: O=C(CS(=O)(=O)c1ccc2nn[nH]c2c1)N1CCN(C(=O)OCc2cc(C(F)(F)F)cc(C(F)(F)F)c2)CC1] | (E) |

### Compound Synthesis

The synthesis of the compounds of the present invention may comprise one or more of the following steps:

### Step 1:

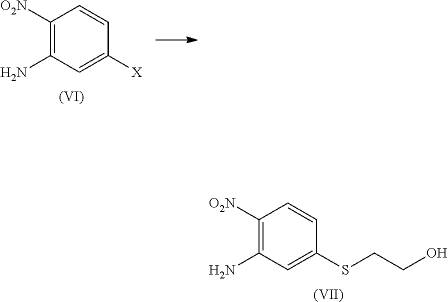

Wherein X is a leaving group. Preferably, X is a sulfonate or a halogen. More preferably, the halogen is selected from the group Cl, I, Br and F, most preferably F. More preferably, the sulfonate is selected from the group consisting mesylate (methylsulfonate), tosylate (p-toluenesulfonate), triflate (trifluoromethylsulfonate).

Compound (VI) is converted with 2-mercaptoethanol and a base. Preferably, the base is a weak base, such as K₂CO₃ or Na₂CO₃.

Preferably, the reaction is carried out at elevated temperature, such 80 to 200° C., more preferably, 90 to 150° C., most preferably 100 to 120° C.

Preferably, the reaction is carried out in a polar-aprotic solvent. More preferably, a polar-aprotic solvent which supports the preferred elevated reaction temperarures of the reaction, such as 1,4-dioxane.

### Step 2:

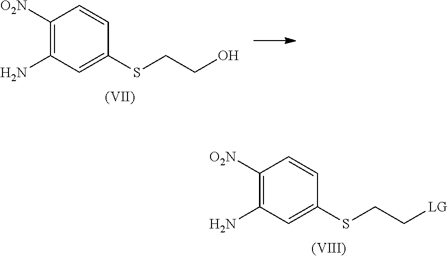

In step 2, the terminal hydroxyl group of (VII) is converted into a leaving group LG.

Preferably, the leaving group LG is a sulfonate or a halogen. More preferably, the halogen is selected from the group Cl, I, Br and F. More preferably, the sulfonate is selected from the group consisting mesylate (methylsulfonate), tosylate (p-toluenesulfonate), triflate (trifluoromethylsulfonate).

Methods for converting hydroxyl groups into leaving groups, in particular sulfonates are known in the art, see for example Peter G. M. Wuts, Greene's Protective Groups in Organic Chemistry, Fifth Edition, Wiley 2014.

### Step 3:

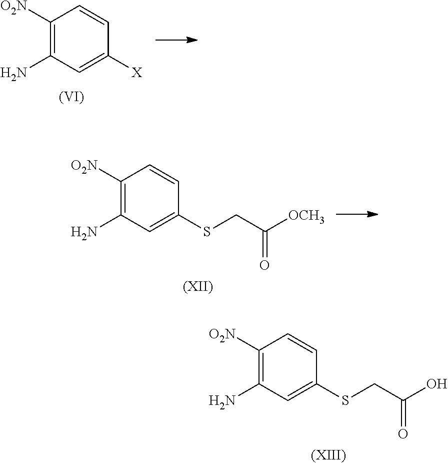

Step 3 may be carried out if J is C═O in Formula (I).

Step 3 represents an alternative to step 1.

Compound (VI) may be converted with methyl thioglycolate and a base into compound (XII). Preferably, the base is a weak base, such as K₂CO₃ or Na₂CO₃.

Compound (XII) may be converted into compound (XIII) by hydrolysis of the ester group with conventional methods known to the person skiled in the art. Preferably, a strong inorganic base such as LiOH, NaOH and KOH is employed, more preferably in the presence of water and/or an organic solvent such as tetrahydrofuran, methanol, ethanol.

### Step 4:

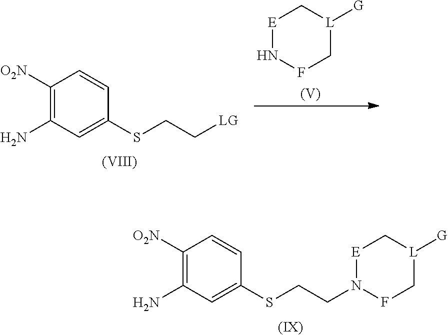

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H; more preferably CH and G is H:
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

In step 4, the LG in (VIII) is substituted by amine (V).

Preferably, the reaction is carried out in the presence of a further base, preferably a weak base, such as i-Pr₂EtN, Et₃N, K₂CO₃, and Na₂CO₃.

Preferably, the reaction is carried out in polar-aprotic solvent, such as THF (tetrahydrofurane), or dioxane.

Preferably, the reaction is carried out at 15 to 100° C., more preferably 50 to 90° C., most preferably 65 to 80° C.

### Step 5:

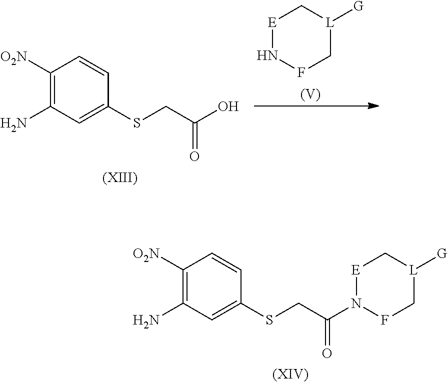

E is CH₂, C═O or CH(C₁-C₅)alkyl; preferably CH₂;
F is CH₂, C═O or CH(C₁-C₅)alkyl; preferably CH₂;
G is H, —C(O)O(C₁-C₅)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

Forming an amide from a carboxylic acid and an amine is known in the art. For example reagents like EDCI (1-Ethyl-3-(3-dimethylaminopropyl) carbodiimide), propylphosphonic anhydride, in the presence of a weak base like NEt₃ and i-Pr₂NEt may be used.

### Step 6:

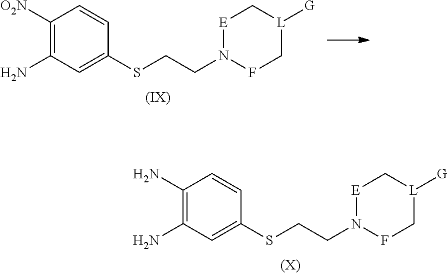

or the same conditions may be applied to compound (XIV) resulting in the corresponding compound

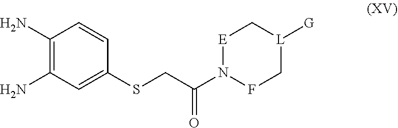

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₅)alkyl, or —C(O)O(CH₂)ₙ(C₅-C₆)aryl; preferably —C(O)O(CH₂)ₙ(C₅-C₆) aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

In step 6, the nitro group in compound (IX) is reduced to the corresponding amino group.

Any suitable agent known to the person in the art may be used for the reduction. Preferably, the reduction is carried out using Na₂S₂O₄.

Preferably, the reaction is carried out in a polar-protic solvent, such as an alcohol and/or water. More preferably, ethanol and/or water.

Preferably, the reaction is carried out at elevated temperature, such as 20 to 100° C., more preferably 40 to 90° C., most preferably, 50 to 80° C.

### Step 7:

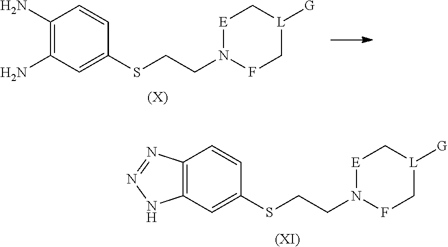

or the same conditions may be applied to compound (XIV) resulting in the corresponding compound

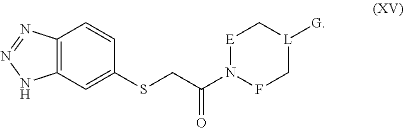

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₅)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

In step 5, both amino groups in compound (IX) are cyclized to a five membered ring.

Preferably, the cyclization is carried out using a NO₂⁻ comprising agent, such as NaNO₂.

Preferably, the reaction is carried out in an acidic solvent, such as acetic acid, in particular glacial acetic acid.

Preferably, the reaction is carried out at 15 to 50° C., more preferably 18 to 30° C., most preferably 20° C.

### Step 8:

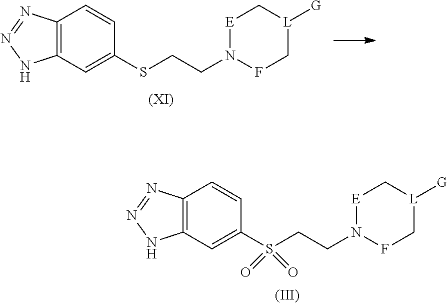

or the same conditions of step 8 may be applied to compound (XV) resulting in the corresponding compound

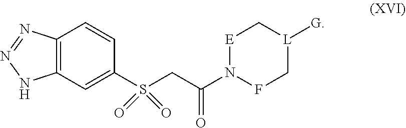

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

Oxidation of the thioether (XI) to the corresponding sulfonyl compound (III) may be achieved in two alternative pathways.

a) Oxidation with mCPBA

It has been found, as demonstrated in the examples, that oxidation with mCPBA may result, besides oxidation of the thioether as desired, in oxidation of the nitrogen in the 6-membered ring and elimination of the 6-membered ring.

As result of the elimination, in case compound (XI) is used as starting material, compound (IV) may be formed.

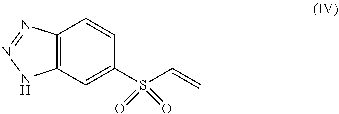

Compound (IV) may be reacted with

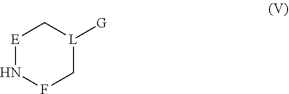

in order to obtain compound (III).
wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

Oxidation/elimination and subsequent reaction with compound (V) may be carried out in one-pot.

Preferably, the reaction is carried out in unpolar aprotic solvents, such as dichloromethane.

The invention comprises a process, preferably for the preparation of (III) comprising

a) The Step of Converting Compound (II) into Compound (III)

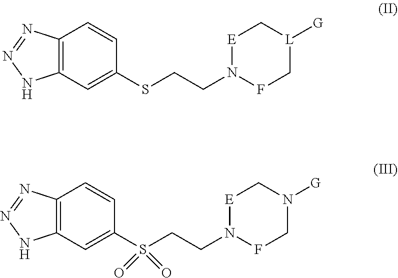

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl; and/or

### b) Formation of Compound (IV)

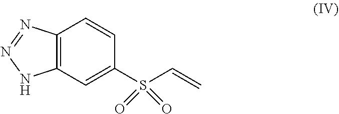

and/or

### c) Formation of Compound (IV) and Addition of Compound (V) at the Vinyl Group of Compound (IV).

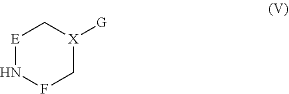

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₅)alkyl, or —C(O)O(CH₂)ₙ(C₅-C₆)aryl; preferably —C(O)O(CH₂)ₙ(C₅-C₆) aryl;
L is N or CH, preferably N or CH and G is H;
n is 1-4, preferably 1-3, more preferably 1
   b) Alternatively, the required oxidation may be carried out applying (NH₄)₆Mo₇O₂₄·4H₂O and H₂O₂.

It has been observed that applying (NH₄)₆Mo₇O₂₄—4H₂O and H₂O₂, preferably does not oxidize the nitrogen of the 6-membered ring and preferably does not induce elimination and formation of compound (IV) to a practically relevant degree.

Preferably, the oxidation is carried out in an acidic solvent, such as acetic acid, preferably glacial acetic acid.

### Step 8

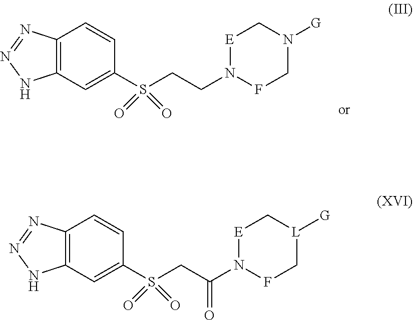

wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
optionally, aryl may be substituted with one or more substituents selected from the group consisting of —CF₃, halogen, —OCF₃, —SCF₃, and (C₁-C₆) alkyl.

Within (III), G may be exchanged, for example from —C(O)O(C₁-C₆)alkyl to —C(O)O(CH₂)ₙ(C₅-C₆)aryl.

If in —C(O)O(C₁-C₆)alkyl, alkyl is tert-butyl, the —C(O)O(C₁-C₆)alkyl group may be replaced by hydrogen under acidic conditions, such as HCl-dioxane. General conditions to add and remove protecting groups are disclosed for example in Peter G. M. Wuts, Greene's Protective Groups in Organic Chemistry, Fifth Edition, Wiley 2014.

As soon as G is hydrogen, applying CDI (carbonyldiimidazole), in combination with a base and HO(CH₂)ₙ(C₅-C₆)aryl allows conversion of (III) into a G wherein G is —C(O)O(CH₂)ₙ(C₅-C₆) aryl.

The base may be selected from the group consisting of NEt₃, and iPr₂NEt, preferably NEt₃.

### Pharmaceutical Compositions

The invention is further directed to a pharmaceutical composition comprising the compound of general formula (I) and at least one pharmaceutically acceptable carrier.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the therapeutic, preferably in purified form, together with a suitable amount of carrier so as to provide the form for proper administration to the patient. The formulation should suit the mode of administration.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate enteral compositions in dosage unit form for ease of administration and uniformity of dosage. Dosage unit form as used herein refers to physically discrete units suited as unitary dosages for the individuals to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

### Medical Use

The compound according to the general formula (I) and the pharmaceutical composition may be for use in medicine.

The invention is further related to the compound according to general formula (I) or the pharmaceutical composition for use in the prevention or in the treatment of diseases in a subject, in which the inhibition, regulation and/or modulation of autotaxin plays a role, preferably comprising reducing reducing the level of lysophosphatidic acid (LPA) in the targeted tissue of said subject, more preferably in the brain of said subject.

The invention is further related to the compound according to general formula (I) or the pharmaceutical composition for use in the prevention or in the treatment of a central nervous system disorder in a subject, comprising reducing the level of lysophosphatidic acid (LPA) in the brain of said subject, a fibrotic disease or in the prevention or treatment of cancer.

As also discussed above, reducing the level of lysophosphatidic acid (LPA) is preferably achieved by inhibition of the protein autotaxin (ATX) which synthesizes LPA, by the compounds of the present invention.

Preferably, the central nervous system disorder is a psychiatric disorder, more preferably the psychiatric disorder is selected from the group consisting of schizophrenia, depression, anxiety disorders, susceptibility to stress and stress-related disorders, panic disorders, bipolar disorder, eating disorders and ADHD, most preferably the psychiatric disorder is obesity or an eating disorder leading to obesity.

Preferably, the eating disorder is binge eating disorder

Preferably, the central nervous system disorder is a neurological disorder, more preferably the neurological disorder is selected from the group consisting of multiple sclerosis, epilepsy, Alzheimer's disease and ischemic stroke.

Preferably, the cancer is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiosarcoma, rhabdomyosarcoma, colon, carcinoma, pancreatic cancer, breast cancer, ovarian cancer, prostate cancer, squamous cell carcinoma, basal cell carcinoma, adenocarcinoma, syringe-carcinoma, sebaceous gland carcinoma, papillary carcinoma, papillary adenocarcinomas, cystadenocarcinomas, bone marrow carcinoma, bronchogenic carcinoma, renal cell carcinoma, hepatoma, bile duct carcinoma, choriocarcinoma, seminoma, embryonic carcinoma, Wilm's tumour, cervical cancer, testicular tumour, lung carcinoma, small-cell lung carcinoma, bladder carcinoma, epithelial carcinoma, glioma, astrocytoma, medulloblastoma, craniopharyngioma, ependyoma, pinealoma, haemangioblastoma, acoustic neuroma, oligodendroglioma, meningioma, melanoma neuroblastoma, retinoblastoma, leukaemia, lymphoma, multiple myeloma, Waldenstrom's macroglobulinaemia and heavy chain disease.

Preferably, the fibrotic disease is selected from the group consisting of, idiopathic lung fibrosis and liver fibrosis.

A better understanding of the present invention and of its advantages will be had from the following examples, offered for illustrative purposes only. The examples are not intended to limit the scope of the present invention in any way.

### EXAMPLES OF THE INVENTION

### Example 1: Synthesis of compound (A)-Route 1

The synthesis of the target compound (A) started by aromatic nucleophilic substitution reaction of 2-mercaptoethanol and 5-fluoro-2-nitroaniline 1 using K₂CO₃ as a base at 110° C. in 1,4-dioxane as a solvent to produce the alcohol 2 in 97% yield.

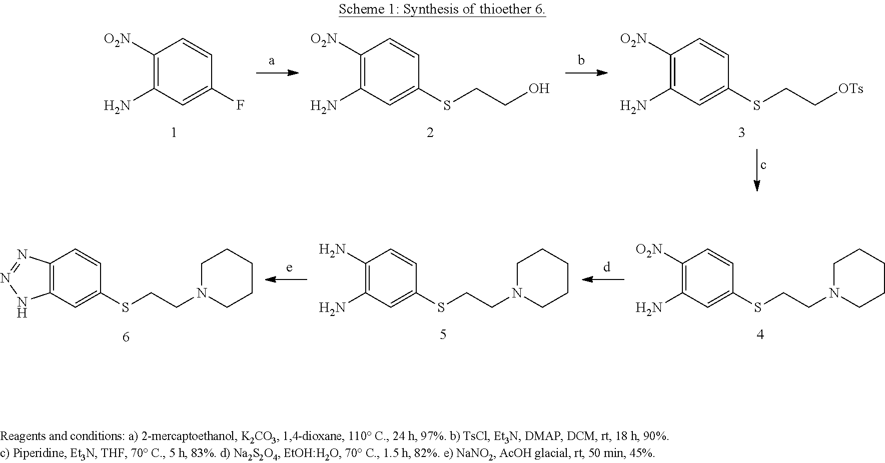 [SMILES: Nc1cc(F)ccc1[N+](=O)[O-] ; Nc1cc(SCCO)ccc1[N+](=O)[O-] ; Cc1ccc(S(=O)(=O)OCCSc2ccc([N+](=O)[O-])c(N)c2)cc1 ; c1cc2nn[nH]c2cc1SCCN1CCCCC1 ; Nc1ccc(SCCN2CCCCC2)cc1N ; Nc1cc(SCCN2CCCCC2)ccc1[N+](=O)[O-]]

Tosylation of alcohol 2 and subsequent substitution with piperidine in excess afforded the tertiary amine 4. The nitro group of 4 was sucessfully reduced to the corresponding phenylendiamine 5 using sodium dithionite in excess as reducing agent in a mixture of ethanol and water at 70° C. Compound 5 tends to oxidize at rt, therefore it is used as it is without any further purification in the next step.

Formation of benzotriazole 6 was performed by adding sodium nitrite to a solution 5 in glacial acetic acid as a solvent. After addition, evolution of gas is observed.

Benzotriazole 6, underwent a one-pot oxidation-elimination-addition reaction to afford product 9 in 54% yield (Scheme 2). First, 6 was over-oxidized to the sulfone-N-oxide intermediate 7 which undergoes a Cope-type elimination to produce the reactive vinyl sulfone 8which is then trapped by the addition of N-boc-piperazine.

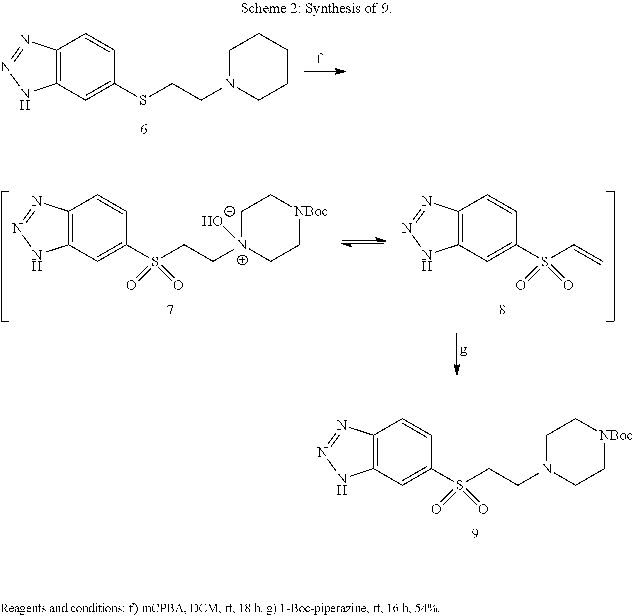

N-Boc cleavage with HCl in dioxane afforded the amine 10 in quantitative yield. Then condensation of 10 with the corresponding benzyl alcohol in the presence of carbonyldiimidazole under basic conditions afforded the target compound in 26% yield (Scheme 3). The yield increased up to 90% if the reaction was carried out for >24 h.

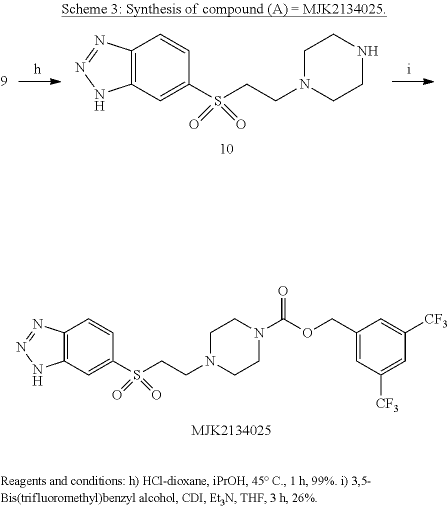 [SMILES: O=S(=O)(CCN1CCNCC1)c1ccc2nn[nH]c2c1 ; O=C(OCc1cc(C(F)(F)F)cc(C(F)(F)F)c1)N1CCN(CCS(=O)(=O)c2ccc3nn[nH]c3c2)CC1]

### Synthesis of 2-((3-amino-4-nitrophenyl)thio)ethan-1-ol (2)

2-mercapto ethanol (9.02 mL, 128.1 mmol) was added to a suspension of 5-fluoro-2-nitroaniline (10 g, 64.05 mmol) and K₂CO₃ (17.71 g, 128.11 mmol) in 1,4-dioxane and heated to 110° C. After 24 h, some 5-fluoro-2-nitroaniline remains unreacted, additional 2-mercaptoethanol (3 mL, 0.25 eq) was added and the reaction kept at 110° C. for another 10 h. The reaction while still warm was filtered; the filter cake is washed with EtOAc until the filtrate was colorless. The solvent is removed under vacuum to afford an orange solid (13 g, 95%). This solid was not purified any further. The compound can be recrystallized from EtOAc.

Exact mass (ESI): m/z=215.0484 (calcd. 215.0485 for C₈H₁₀N₂O₃S [M+H]⁺).

¹H NMR (300 MHz, DMSO) δ 7.85 (d, J=9.1 Hz, 1H), 7.44 (s, 2H, NH₎, 6.86 (d, J=2.1 Hz, 1H), 6.50 (dd, J=9.1, 2.1 Hz, 1H), 5.05 (s, broad, 1H, OH), 3.63 (t, J=6.6 Hz, 2H), 3.08 (t, J=6.6 Hz, 2H).

### Synthesis of 2-((3-amino-4-nitrophenyl)thio)ethyl 4-methylbenzenesulfonate (3)

Under N₂ TsCl (4.45 g, 30.3 mmol) was added in one portion to a solution of 2 (5 g, 23.3), Et3N (2.4 g, 30.3 mmol) and DMAP (0.29 g, 2.3 mmol) in dry DCM and stirred at rt for 18 h. The reaction mixture is diluted with DCM and washed with NaHCO₃ solution, brine, and water. The organic layer was dried (Na₂SO₄) and the solvent was removed under reduced pressure to afford a yellow oil. The compound was used in the next step without further purification.

Exact mass (ESI): m/z=369.0570 (calcd. 369.0573 for C₁₅H₁₇N₂O₅S₂ [M+H]⁺).

¹H NMR (300 MHz, CDCl₃) δ 7.96 (d, J=9.0 Hz, 1H), 7.79 (d, J=8.3 Hz, 2H), 7.75 (d, J=8.4 Hz, 1H), 7.35 (d, J=8.8 Hz, 2H), 7.31 (s, 1H), 5.86 (s, broad, 2H, NH₂), 4.21 (t, J=6.9Hz, 2H), 2.84 (t, J=6.6 Hz, 2H), 2.45 (s, 3H).

### Synthesis of 2-nitro-5-((2-(piperidin-1-yl)ethyl)thio)aniline (4)

Piperidine (2.4 g, 28 mmol) was added to a solution of tosylate 3 (3.4 g, 9.33 mmol) in THF and heated to 45° C. After 3 h, one major more polar compound was observed by TLC, additional piperidine (1 mL) was added to the mixture at 45° C. The reaction was completed after 24 h. The reaction mixture was cooled down to rt and poured into water, extracted with ethyl acetate (3×15 mL). The combined organic layers were dried (Na₂SO₄), the solvent removed under reduced pressure, redissolved in DCM, and adsorbed on silica gel for further purification by flash chromatography. Yellow resin (2.2 g, 83%).

Exact mass (ESI): m/z=282.1266 (calcd. 282.1271 for C₁₃H₂₀N₃O₂S [M+H]⁺).

¹H NMR (300 MHz, CDCl₃) δ 7.99 (d, J=9.0 Hz, 1H), 6.59 (d, J=1.9 Hz, 1H), 6.54 (dd, J=9.0, 2.0 Hz, 1H), 6.13 (s, 2H), 3.13-3.04 (m, 5H), 2.87-2.78 (m, 5H), 2.67-2.57 (m, 6H).

### Synthesis of 4-((2-(piperidin-1-yl)ethyl)thio)benzene-1,2-diamine (5)

A dispersion of sodium dithionite (3.7 g, 21.3 mmol) in water was added to a previously heated (70° C.) ethanolic solution of 4 (1.0 g, 3.6 mmol) and stirred at 70° C. for 1 h. The reaction mixture was filtered, the filter cake washed with ethanol and the ethanol was removed under reduced pressure, and the residue was diluted with NaOH 1M and extracted with DCM (6×20 mL), the combined organic layers were dried (Na₂SO₄), and the solvent was removed under reduced pressure. Pale yellow solid resin (731 mg, 82%).

Exact mass (ESI): m/z=252.1526 (calcd. 252.1529 for C₁₃H₂₂N₃S [M+H]⁺).

### Synthesis 6-((2-(piperidin-1-yl)ethyl)thio)-1H-benzo[d][1,2,3]triazole (6)

NaNO₂ (220.4 mg, 3.2 mmol) was added to a solution of 5 (731 mg, 2.9 mmol) in glacial acetic acid (10 mL) at rt. After addition a change in color from yellow to orange and evolution of gas was noticed. After 50 min the reaction was stopped. The reaction mixture was diluted with 1 M NaOH (40 mL) and the pH adjusted to PH˜8 slowly in an ice bath with Na₂CO₃, then extracted with DCM (3×20 mL), the combined organic layers were dried (Na₂SO₄), the solvent removed under reduced pressure, redissolved in DCM and adsorbed on silica gel for further purification. The crude residue was adsorbed on a small amount of silica gel/Celite and purified via column chromatography.

The target compound is isolated as a yellow solid in 45% yield (340 mg).

Exact mass (ESI): m/z=263.1318 (calcd. 262.1325 for C₁₃H₁₉N₄S [M+H]⁺).

¹H NMR (500 MHz, CDCl₃) δ 12.35 (s, 1H), 7.81 (dd, J=1.6, 0.8 Hz, 1H), 7.59 (dd, J=8.6, 0.8 Hz, 1H), 7.21 (dd, J=8.7, 1.6 Hz, 1H), 3.23-3.16 (m, 2H), 2.82-2.76 (m, 2H), 2.67-2.61 (m, 4H), 1.67 (p, J=5.7 Hz, 4H), 1.48 (t, J=5.8 Hz, 2H).

¹³C NMR (126 MHz, CDCl₃) δ 139.64, 139.10, 133.37, 127.53, 115.82, 115.09, 57.69, 54.14, 30.78, 25.29, 25.16, 25.07, 23.83.

### Synthesis of tert-butyl 4-(2-((1H-benzo [d][1,2,3]triazol-6-yl)sulfonyl)ethyl)piperazine-1-carboxylate (9)

mCPBA (564 mg, 3.3 mmol) was added to a solution of 6 (245, mg, 0.9 mmol) in DCM (25 mL) at rt. After 2 h a colorless precipitate was formed. N-Boc-piperazine (608.3 mg, 3.3mmol) was added to the reaction mixture in one portion, and the reaction was kept for 18 h at rt. After 18 h the reaction mixture is washed with NaHCO₃ solution (3×15 mL), the organic layer was dried over Na₂SO₄, adsorbed on silica gel for further purification by flash chromatography. White solid (200 mg, 54%).

Exact mass (ESI): m/z=396.1695 (calcd. 396.1700 for C₁₇H₂₆N₅O₄S [M+H]⁺).

¹H NMR (500 MHz, MeOD) δ 8.59 (dd, J=1.5, 0.9 Hz, 1H), 8.06 (dd, J=8.7, 0.9 Hz, 1H), 8.02 (dd, J=8.7, 1.5 Hz, 1H), 3.55 (t, J=6.8 Hz, 2H), 3.11 (t, J=5.1 Hz, 4H), 2.77 (t, J=6.8 Hz, 2H), 2.30-2.25 (m, 4H), 1.40 (s, 9H).

¹³C NMR (126 MHz, MeOD) δ 156.21, 138.52, 133.64, 131.08, 126.34, 119.86, 115.61, 81.25, 54.14, 53.34, 52.64, 28.58.

### Synthesis of 6-((2-(piperazin-1-yl)ethyl)sulfonyl)-1H-benzo[d][1,2,3]triazole (10)

HCl-Dioxane (5 mL) was added to a solution of sulfone 9 (200 mg, 0.51 mmol) in iPrOH at rt. The reaction was stirred overnight at 40° C. The solvent was removed under reduced pressure to afford a yellow solid. The product was used without any further purification in the next step.

Exact mass (ESI): m/z=296.1175 (calcd. 296.1176 for C₁₂H₁₈N₅O₂S [M+H]⁺).

### Synthesis of 3,5-bis(trifluoromethyl)benzyl 4-(2-((1H-benzo[d][1,2,3]triazol-6-yl)sulfonyl)ethyl)piperazine-1-carboxylate (A)

CDI (91 mg, 0.56 mmol) was added to a solution of 3,5-bis(trifluoromethyl)benzyl alcohol (316 mg, 0.56 mmol) in THF (10 mL) at rt and stirred for 3 h. The amine 10 (150 mg, 0.51 mmol) and Et₃N (176 μL, 1.26 mmol)) were dissolved in THF (9 mL) and DMF (2 mL) and stirred at rt. The benzyl alochol CDI reaction was added to this solution, and the mixture was stirred at 40° C. for 2 h. The reaction mixture was poured into cold water and extracted with ethyl acetate and NaCl was added until saturation. The combined organic layers were dried with Na₂SO₄ and evaporated to dryness. Purification by flash chromatography yielded a white solid (75 mg, 26%).

Exact mass (ESI): m/z=566.1302 (calcd.566.1291 for C₂₂H₂₂F₆N₅O₄S [M+H]⁺).

¹H NMR (500 MHz, MeOD) δ 8.59 (t, J=1.2 Hz, 1H), 8.04 (dd, J=8.8, 0.9 Hz, 1H), 8.01 (dd, J=8.7, 1.5 Hz, 1H), 7.88 (s, 2H), 7.87 (s, 1H), 5.21 (s, 2H), 3.52 (t, J=6.8 Hz, 2H), 3.29-3.19 (m, 4H), 2.78 (t, J=6.8 Hz, 2H), 2.32 (t, J=5.1 Hz, 4H).

¹³C NMR (126 MHz, MeOD) δ 155.93, 141.23 (2C), 138.17, 132.79 (q, J=33.3 Hz), 129.07 (q, J=3.9 Hz), 126.11, 124.48 (q, J=271.9 Hz), 122.66 (p, J=3.9 Hz), 119.75, 115.51, 66.50, 54.08, 53.14, 52.45, 44.53.

Purity (UHPLC): 99.02% (t_{R}=3.95 min).

### Example 1: Synthesis of Compound (A)-Route 2

The synthesis of the target compound started by substitution of the tosylate 3 with 1-boc-piperazine to afford 11. Nitroreduction with sodium dithionite as reducing agent and subsequent reaction with sodium nitrite affords the triazole 13. N-Boc deprotection with HCl-Dioxane solution in isopropanol affords piperazine 14 in quantitative yield (Scheme 4).

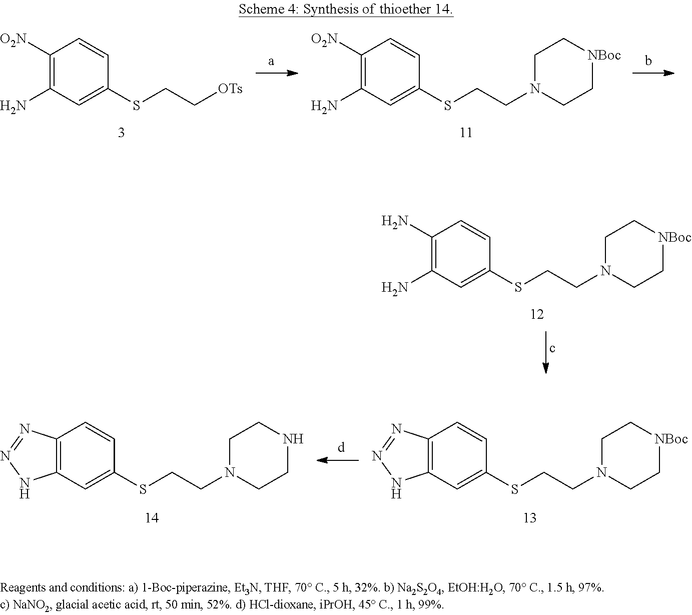 [SMILES: Cc1ccc(S(=O)(=O)OCCSc2ccc([N+](=O)[O-])c(N)c2)cc1 ; CC(C)(C)OC(=O)N1CCN(CCSc2ccc([N+](=O)[O-])c(N)c2)CC1 ; CC(C)(C)OC(=O)N1CCN(CCSc2ccc(N)c(N)c2)CC1 ; c1cc2nn[nH]c2cc1SCCN1CCNCC1 ; CC(C)(C)OC(=O)N1CCN(CCSc2ccc3nn[nH]c3c2)CC1]

Carbamate formation with the corresponding benzyl alcohol and 14 and CDI as coupling reagent results in thioether 15 in 90%. Finally, oxidation of 15 is performed by adding hydrogen peroxide stepwise in glacial acetic acid as solvent. Acidic conditions are required to avoid competing N-oxidation reaction (Scheme 5).

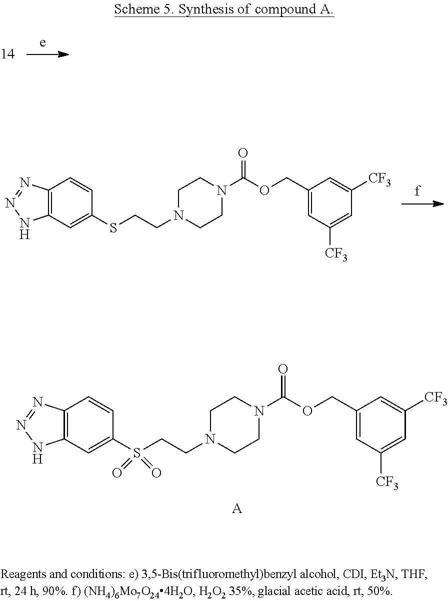

### Synthesis of tert-butyl 4-(2-((3-amino-4-nitrophenyl)thio)ethyl)piperazine-1-carboxylate (11)

1-boc-piperazine (1.6 g, 8.8 mmol) was added to a solution of tosylate 3 (1.7 g, 5.9 mmol) in THF and heated to 70° C. After 5 h the reaction mixture was cooled down to room temperature and poured into water and extracted with ethyl acetate (3×15 mL). The combined organic layers were dried (Na₂SO₄), the solvent removed under reduced pressure, redissolved in DCM, and adsorbed on silica gel for further purification by flash chromatography. Yellow powder (0.7 g, 32%).

Exact mass (ESI): m/z=383.1741 (calcd. 383.1748 for C₁₇H₂₆N₄O_{4S [M+H]}⁺).

¹H NMR (300 MHz, CDCl₃) δ 8.01 (d, J=9.0 Hz, 1H), 6.59 (d, J=2.0 Hz, 1H), 6.55 (dd, J=9.0, 2.0 Hz, 1H), 6.16 (s, 2H), 3.48-3.42 (m, 4H), 3.14-3.07 (m, 2H), 2.74-2.64 (m, 2H), 2.45 (t, J=5.1 Hz, 4H), 1.46 (s, 9H).

### Synthesis of tert-butyl 4-(2-((3,4-diaminophenyl)thio)ethyl)piperazine-1-carboxylate (12)

A dispersion of sodium dithionite (1.9 g, 11.0 mmol) in water was added to a previously heated (70° C.) ethanolic solution of 11 (0.7 g, 1.8 mmol) and stirred at 70° C. for 1 h. The reaction mixture was filtered, the filter cake washed with ethanol and the ethanol is removed under vacuum, and the residue was diluted with NaOH 1M and extracted with DCM (6×20 mL), the combined organic layers were dried (Na₂SO₄) and the solvent was removed under reduced pressure. Pale yellow solid resin (623 mg, 97%).

Exact mass (ESI): m/z=375.1822 (calcd. 375.1825 for C₁₇H₂₈N₄O₂S [M+H]⁺).

¹H NMR (300 MHz, CDCl₃) δ 6.81-6.76 (m, 2H), 6.63-6.59 (m, 1H), 3.40 (t, J=5.1 Hz, 4H), 2.93-2.86 (m, 2H), 2.62-2.51 (m, 2H), 2.38 (t, J=5.1 Hz, 4H), 1.44 (s, 9H).

¹³C NMR (75 MHz, CDCl₃) δ 154.84, 135.26, 134.45, 125.18, 124.27, 120.41, 117.13, 79.76, 58.19, 52.97, 33.01, 28.55.

### Synthesis of tert-butyl 4-(2-((1H-benzo[d][1,2,3]triazol-6-yl)thio)ethyl)piperazine-1-carboxylate (13)

NaNO₂ (119 mg, 1.7 mmol) was added to a solution of 12 (600 mg, 1.7 mmol) in glacial acetic acid (10 mL) at rt. After 50 minutes, the reaction mixture was diluted with 1 M NaOH (40 mL) and the pH adjusted to PH˜8 slowly using an ice bath with Na₂CO₃, then extracted with DCM (3×20 mL), the combined organic layers were dried (Na₂SO₄), the solvent removed under reduced pressure, redissolved in DCM, and adsorbed on silica gel for further purification. The crude residue was adsorbed on a small amount of silica gel purified via column chromatography. Yellow vitreous solid, (322 mg, 52%).

Exact mass (ESI): m/z=364.1792 (calcd. 364.1802 for C₁₇H₂₅N₅O₂S [M+H]⁺).

¹H NMR (300 MHz, CDCl₃) δ 7.82 (dd, J=8.7, 0.8 Hz, 1H), 7.78 (s, 1H), 7.37 (dd, J=8.7, 1.6 Hz, 1H), 3.47 (t, J=5.0 Hz, 4H), 3.19-3.09 (m, 2H), 2.75-2.67 (m, 2H), 2.47 (t, J=5.0 Hz, 4H), 1.47 (s, 9H).

¹³C NMR (75 MHz, CDCl₃) δ 155.08, 139.22, 138.59, 135.51, 127.58, 116.40, 113.07, 80.26, 57.45, 52.94, 31.12, 28.58.

### Synthesis of 6-((2-(piperazin-1-yl)ethyl)thio)-1H-benzo[d][1,2,3]triazole (14)

HCl-Dioxane (5 mL) was added to a solution of thioether 13 (310 mg, 0.9 mmol) in iPrOH at 45° C. After 1 h, the solvent was removed under reduced pressure to afford a yellow solid (224 mg, 99%). The product was used without any further purification in the next step.

Exact mass (ESI): m/z=264.1275 (calcd. 264.1277 for C₁₂H₁₇N₅S [M+H]⁺).

### Synthesis of 3,5-bis(trifluoromethyl)benzyl 4-(2-((1H-benzo [d][1,2,3]triazol-6-yl)thio)ethyl)piperazine-1-carboxylate (15)

CDI (68 mg, 0.4 mmol) was added to a solution of 3,5-bis(trifluoromethyl)benzyl alcohol (103 mg, 0.4 mmol) in THF at rt and stirred for 3 h. The amine 14 (224 mg, 0.4 mmol) and Et₃N (85 mg, 0.8 mmol)) were dissolved in THF (9 mL) and DMF (2 mL) and stirred at rt. The benzyl alcohol-CDI reaction was added to this solution, and the mixture was stirred at rt for 24 h. The reaction was poured into cold water and extracted with ethyl acetate, the combined organic layers were dried with Na₂SO₄ and evaporated to dryness. Purification by flash chromatography yielded a white solid (201 mg, 26%).

Exact mass (ESI): m/z=534.1388 (calcd. 534.1393 for C₂₂H₂₁F₆N₅O₂S [M+H]⁺).

¹H NMR (500 MHz, CDCl₃) δ 8.05 (s, 1H), 7.82-7.77 (m, 4H), 7.37 (dd, J=8.7, 1.5 Hz, 1H), 5.22 (s, 2H), 3.54 (t, J=5.1 Hz, 4H), 3.15-3.10 (m, 2H), 2.74-2.69 (m, 2H), 2.50 (t, J=5.0 Hz, 4H).

¹³C NMR (126 MHz, CDCl₃) δ 154.67, 139.44, 134.87, 132.04 (q, J=33.4 Hz), 127.88 (d, J=3.9 Hz), 127.71, 123.25 (q, J=272.4 Hz), 122.15 (dd, J=7.8, 4.0 Hz), 114.01, 65.65, 57.35, 52.72, 43.90.

### Synthesis of 3,5-bis(trifluoromethyl)benzyl 4-(2-((1H-benzo[d][1,2,3]triazol-6-yl)sulfonyl)ethyl)piperazine-1-carboxylate (A)

H₂O₂ 35% (2.7 μL) was added to a solution of (NH₄)₆Mo₇O₂₄·7H₂O (21 mg, 0.02 mmol), and thioether 15 (30 mg, 0.06 mmol) in glacial acetic acid (2 mL) at rt. After 40 min H₂O₂ 35% (2.7 μL) was added to the solution. After 50 minutes, the reaction was quenched with 2% mercaptoethanol (0.5 mL), the reaction was diluted with NaOH 1 M until pH 7-8, then extracted with DCM (3×10 mL), the combined organic layers were dried Na₂SO₄ and evaporated to dryness. Purification by flash chromatography yielded a white solid (16 mg, 50%).

Exact mass (ESI): m/z=566.1286 (calcd. 566.1291 for C₂₂H₂₂F₆N₅O₄S [M+H]⁺).

Purity (UHPLC): 97.24% (t_{R}=3.93 min).

### Synthesis of 3,5-bis(trifluoromethyl)benzyl 4-(2-((1H-benzo[d][1,2,3]triazol-6-yl)thio)acetyl)piperazine-1-carboxylate (MJK2234001) and 3,5-bis(trifluoromethyl)benzyl 4-(2-((1H-benzo[d][1,2,3]triazol-6-yl)sulfonyl)acetyl)piperazine-1-carboxylate (MJK2234002)

The synthesis of the target compounds started with an aromatic nucleophilic substitution reaction of methyl thioglycolate and 5-fluoro-2-nitroaniline (1) using K₂CO₃ as a base at 95° C. in 1,4-dioxane to produce the thioether 16 (Scheme 6).

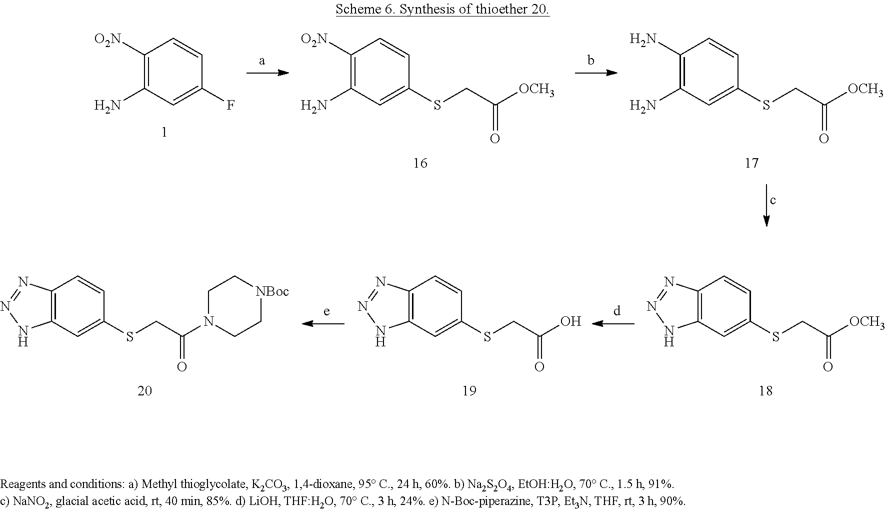 [SMILES: Nc1cc(F)ccc1[N+](=O)[O-] ; COC(=O)CSc1ccc([N+](=O)[O-])c(N)c1 ; COC(=O)CSc1ccc(N)c(N)c1 ; CC(C)(C)OC(=O)N1CCN(C(=O)CSc2ccc3nn[nH]c3c2)CC1 ; O=C(O)CSc1ccc2nn[nH]c2c1 ; COC(=O)CSc1ccc2nn[nH]c2c1]

Reduction to the corresponding phenylenediamine 17 with sodium dithionite followed by reaction with sodium nitrite in acidic media resulted in the benzotriazole 18.Compound 18 was hydrolyzed under basic conditions to carboxylic acid 19 which upon activation with propylphosphonic anhydride (T3P) and subsequent reaction with N-boc-piperazine afforded compound 20.

N-Boc cleavage with HCl in dioxane afforded the amine 21 in 92% yield. Then condensation of 21 with the corresponding benzyl alcohol in the presence of carbonyldiimidazole under basic conditions afforded MJK2234001 in 61% yield (Scheme 7).

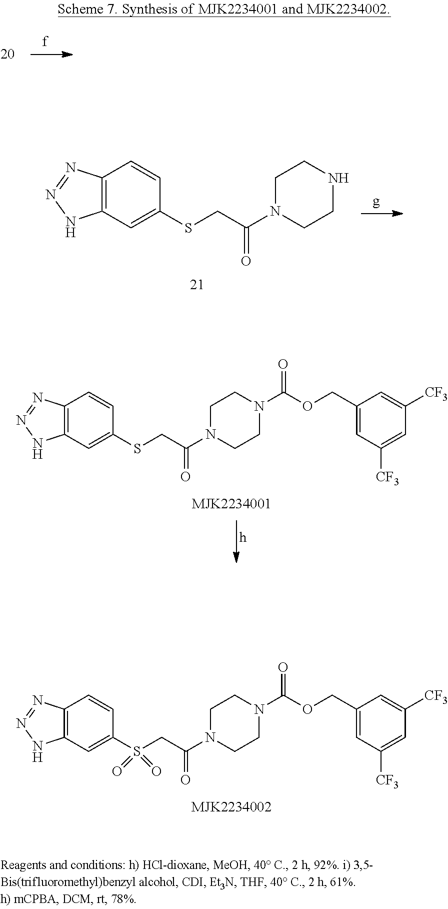 [SMILES: O=C(CSc1ccc2nn[nH]c2c1)N1CCNCC1 ; O=C(CSc1ccc2nn[nH]c2c1)N1CCN(C(=O)OCc2cc(C(F)(F)F)cc(C(F)(F)F)c2)CC1 ; "MJK2234001" ; O=C(CS(=O)(=O)c1ccc2nn[nH]c2c1)N1CCN(C(=O)OCc2cc(C(F)(F)F)cc(C(F)(F)F)c2)CC1 ; "MJK2234002"]

The two-step oxidation of thioether MJK2234001 to the target sulfone MJK2234002 was performed by portion-wise addition of m-chloroperbenzoic acid (mCPBA) to the reaction mixture.

### Synthesis of methyl 2-((3-amino-4-nitrophenyl)thio)acetate (16)

Methyl thioglycolate (4.3 mL, 48.03 mmol) is added to a suspension of 5-fluoro-2-nitroaniline (5 g, 32.03 mmol) and K₂CO₃ (7.97 g, 57.65 mmol) in 1,4-dioxane and heated to 95° C. for 24 h. The warm reaction mixture was filtered through a filter paper; the filter cake was washed with ethyl acetate until the filtrate was colorless. The solvent was removed under reduced pressure to afford a yellow solid (4.6 g, 60%). This solid was not purified further and used for further steps.

¹H NMR (300 MHz, CDCl₃) δ 8.02 (d, J=9.0 Hz, 1H), 6.68 (d, J=2.0 Hz, 1H), 6.58 (dd, J=9.0, 2.1 Hz, 1H), 6.15 (s, 2H), 3.77 (s, 3H), 3.72 (s, 2H).

¹³C NMR (75 MHz, CDCl₃) δ 169.41, 146.31, 144.97, 130.33, 126.85, 115.33, 114.51, 53.11, 34.21.

### Synthesis of methyl 2-((3,4-diaminophenyl)thio)acetate (17)

A dispersion of sodium dithionite (8.9 g, 51.60 mmol) in water was added to a previously heated (70° C.) ethanolic solution of 16 (2.5 g, 10.32 mmol) and maintained at 70° C. for 1 h, after this time the solution changed from intense yellow to pale yellow. TLC revealed the starting material was fully consumed and a polar product was most abundant. The reaction mixture was filtered, the filter cake was washed with EtOH, the solvent was removed under reduced pressure, and the residue was diluted with NaOH 1M and extracted with DCM (6×20mL). The combined organic layers were dried (Na₂SO₄), the solvent was removed under reduced pressure and the product was used for the next step without any further purification. Pale yellow solid resin (2.0 g, 91%).

Exact mass (ESI): m/z=213.0687 (calcd. 213.0692 for C_{g}H₁₃N₂O₂S [M+H]⁺).

### Synthesis 6-((2-(piperidin-1-yl)ethyl)thio)-1H-benzo[d][1,2,3]triazole (18)

NaNO₂ (715.0 mg, 10.36 mmol) was added to a solution of 17 (2.0 g, 9.42 mmol) in glacial acetic acid (20 mL) at rt. After addition a change in color from yellow to orange and evolution of gas was noticed, after 50 min TLC (DCM:MeOH 9:1) showed that all the starting material was consumed and one major product was fomed. The reaction mixture was diluted with 1 M NaOH (40 mL) and the pH adjusted to pH˜8 slowly in an ice bath with Na₂CO₃ and extracted with DCM (3×20 mL). The combined organic layers were dried (Na₂SO₄), the solvent was removed under reduced pressure, redissolved in DCM and adsorbed on silica gel for further column chromatographic purification (DCM: (DCM/MeOH 7:3); 1:0→7:3→0:1). The target compound was isolated as a yellow solid in 85% yield (1.8 g).

Exact mass (ESI): m/z=224.0485 (calcd. 224.0489 for C₉H₁₀N₃O₂S [M+H]⁺).

¹H NMR (300 MHz, CDCl₃) δ 14.01 (s, 1H), 7.95 (s, 1H), 7.82 (d, J=8.7 Hz, 1H), 7.44 (dd, J=8.8, 1.6 Hz, 1H), 3.76 (s, 2H), 3.73 (s, 3H).

### Synthesis 2-((1H-benzo[d][1,2,3]triazol-6-yl)thio)acetic acid (19)

LiOH (289.2 mg, 12.07 mmol) was added to a solution of 18 (1.8 g, 8.05 mmol) in a mixture of THF: H₂O (10% v/v, 20 mL) at 60° C. for 3 h. The solution was poured into water, acidified to pH 2 with HCl (1M) and extracted with ethyl acetate (3×15 mL). The combined organic layers were dried (Na₂SO₄), the solvent was removed under reduced pressure to afford a pale yellow solid (411 mg, 24%). This solid was used for the next step without any further purification.

### Synthesis tert-butyl 4-(2-((1H-benzo[d][1,2,3]triazol-6-yl)thio)acetyl)piperazine-1-carboxylate (20)

Propylphosphonic anhydride (T3P, 2.8 mL, 50% wt in EtOAC, 4.58 mmol) was added to a solution of 19 (400 mg, 1.91 mmol) in dry THF and stirred at room temperature for 15 min. Then, triethyl amine (0.67 mL, 4.77 mmol) and N-boc-piperazine (428 mg, 2.29 mmol) were added to the mixture and stirred at rt for 4 h. The solution was poured into water, extracted with ethyl acetate (3×15 mL) and the combined organic layers were washed with a citric acid solution, then with NaOH 1M and dried (Na₂SO₄). The solvent was removed under reduced pressuere to afford a pale colorless solid (647 mg, 90%). The product was used in the next reaction without any further purification.

Exact mass (ESI): m/z=378.1587 (calcd. 378.1594 for C₁₇H₂₄N₅O₃S [M+H]⁺).

### Synthesis of 2-((1H-benzo [d][1,2,3]triazol-6-yl)thio)-1-(piperazin-1-yl)ethan-1-one (21)

HCl-dioxane (5 mL) was added to a solution of 20 (600 mg, 0.51 mmol) in MeOH at the mixture was stirred at 40° C. for 2 h. The solvent was removed under reduced pressure to afford a yellow solid. The product was used without any further purification in the next step. LC-MS analysis showed that the residue was a mixture of the expected product (89%) and the unreacted starting material 20 (11%).

Exact mass (ESI): m/z=278.1063 (calcd. 278.3535 for C₁₂H₁₆N₅OS [M+H]⁺).

### Synthesis of 3,5-bis(trifluoromethyl)benzyl 4-(2-((1H-benzo[d][1,2,3]triazol-6-yl)thio)acetyl)piperazine-1-carboxylate (MJK2234001)

CDI (283.6 mg, 1.75 mmol) was added to a solution of benzyl alcohol (426.9 mg, 1.75 mmol) in THF at room temperature and stirred. After 2.5 h alcohol was almost consumed and the reaction was stirred further 30 minutes. In parallel, the amine 21 (441 mg, 1.59 mmol) and Et₃N (0.55 mL, 3.98 mmol) were dissolved in THF (9 mL) and DMF (2 mL) and stirred at rt. The benzyl alcohol-CDI reaction mixture was added to this solution, and the mixture was stirred at 40° C. for 2 h. LC-MS revealed that all the amine was consumed and major product was formed. The reaction was poured into cold water and NaCl and extracted with ethyl acetate (3×20 mL). The combined organic layers were dried over Na₂SO₄ and the solvents were evaporated. Purification was performed using flash chromatography with petrol ether: (ethyl acetate: MeOH 5%) 1:0 to 0:1 to obtain a pale yellow solid (530 mg, 61%).

Exact mass (ESI): m/z=548.1184 (calcd. 548.1186 for C₂₂H₁₉F₆N₅O₃S [M+H]⁺).

¹H NMR (600 MHz, CDCl₃) δ 8.04 (s, 1H), 7.85 (s, 1H), 7.82 (s, 2H), 7.33 (dd, J=8.6, 1.6 Hz, 1H), 5.27 (s, 2H), 3.86 (s, 2H), 3.69 (s, 4H), 3.65-3.57 (m, 5H).

¹³C NMR (151 MHz, CDCl₃) δ 162.82, 154.65, 138.98, 132.12 (q, J=33.5 Hz), 128.20 (d, J=4.0 Hz), 126.02-120.45 (m), 122.37, 66.05, 46.38, 44.49-43.36 (m), 42.00, 31.65.

Purity (UHPLC): 92.38% (t_{R}=4.39 min)

### Synthesis of 3,5-bis(trifluoromethyl)benzyl 4-(2-((1H-benzo[d][1,2,3]triazol-6-yl)sulfonyl)acetyl)piperazine-1-carboxylate (MJK2234002)

m-Chloroperbenzoic acid (mCPBA, 47 mg, 0.27 mmol) was added stepwise (3 portions) every 20 minutes to a solution of thioether MJK2234001 in DCM at room temperature. After the addition of the last portion, the reaction was stirred until total oxidation of the intermediate sulfoxide to the sulfone. The reaction was terminated by adding a solution of mercaptoethanol in methanol (1%). The reaction was poured into cold water and extracted with ethyl acetate (3×20 mL) and washed with NaOH 1 M solution. The combined organic layers were dried over Na₂SO₄ and the solvent was evaporated. Purification was performed by flash chromatography with petrol ether: (ethyl acetate: MeOH 5%) 1:0 to 0:1 to obtain a colorless solid (41 mg, 78%).

Exact mass (ESI): m/z=580.1079 (calcd. 580.1084 for C₂₂H₁₉F₆N₅O₅S [M+H]⁺).

¹H NMR (500 MHz, DMSO) δ 8.57 (s, 1H), 8.16-8.05 (m, 4H), 7.99-7.94 (m, 1H), 5.27 (s, 2H), 4.83 (s, 2H), 3.62-3.37 (m, 8H).

¹³C NMR (126 MHz, DMSO) δ 162.34, 160.18, 154.13, 140.34, 133.37, 132.77, 130.34 (q, J=32.3 Hz), 128.86, 128.52, 127.96, 123.31 (q, J=272.8 Hz), 121.76, 65.03, 58.37, 45.74, 41.29.

Purity (UHPLC): 97.64% (t_{R}=4.28 min).

### Example 2: ATX Activity Assay

### 2.1 Preparation of a Dilution Series of the Inhibitor

A solution of 10 mM of the inhibitor to test in DMSO was received. Adding 40.5 μL of this solution to 959.5 μL DMSO gave a concentration of 405 μM. Adding 820 μL water to 180 μL of the previous solution yielded a total volume of 960 μL (volume reduction caused by solving DMSO in water) and an inhibitor concentration of 75.94 μM. For a threefold dilution series 800 μL of 18% DMSO in water were dispensed in twelve 1.5 mL test tubes. 400 μL of the 75.9 μM inhibitor solution were added to the first vial and the solution was vortexed. For every dilution step 400 μL of the corresponding previous dilution step were transferred and mixed by vortexing. The obtained concentrations were 7594 nM, 2531 nM, 844 nM, 281 nM, 94 nM, 31 nM, 10 nM, 3.47 nM, 1.16 nM, 0.39 nM, 0.13 nM, 0.043 nM, 0.014 nM. The eight concentration steps from 31 nM to 0.014 nM were used in the assay.

### 2.2 Performing the Autotaxin Inhibitor Test Kit Assay

The assay was performed according to the attached manual of the assay kit. In brief 80 μL reaction buffer containing ATX, 10 μL of the dilution series from the inhibitor and 10 μL fluorescent ATX substrate solution were mixed in a well of a 96 well plate. Cleavage of the substrate by ATX causes increase in fluorescence (excitation 485 nm, emission 528 nm), which is measured by a plate reader in 1 min intervals. The increase of fluorescence between 5 min and 15 min was used to calculate the reaction rate.

The test of quenching of fluorescence of the cleft substrate by the inhibitor showed no quenching and was therefore not included in the calculation.

### 2.3 Statistical Analysis

The autotaxin reaction was performed in four parallel replicates for the target inhibitor and two replicates for the positive control BrP-LPA. The slope of the increase in fluorescence was calculated by Excel using the “slope” function. The IC₅₀ was calculated by GraphPad Prism using the nolinear regression (curve fit): “log (inhibitor) vs. response with variable slope (four parameters)”.

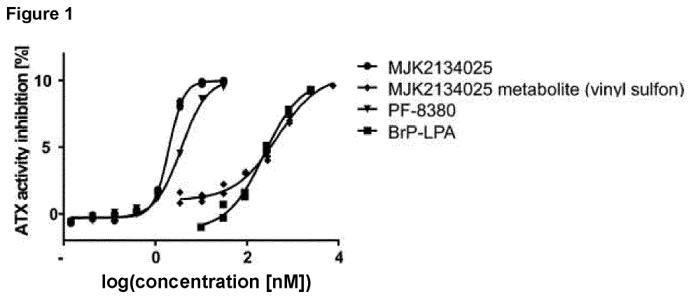

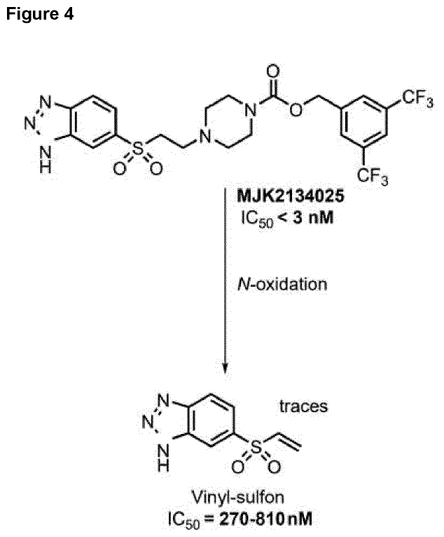

The results are displayed in FIGS. 1 and 4.

### 2.3 Further Biological Testing

TABLE 3
ATX Inhibitory and cytotoxic activity of compounds.

| Compound | IC₅₀ (nM) ATX inhibition | CC₅₀ (nM) in HeLa |
|---|---|---|
| MJK2134025 | 1.8 | 45090 |
| MJK2234001 | 4.0 | 6200 |
| MJK2234002 | 2.0 | 67800 |
| PF-8380 | 4.8 | n.m |

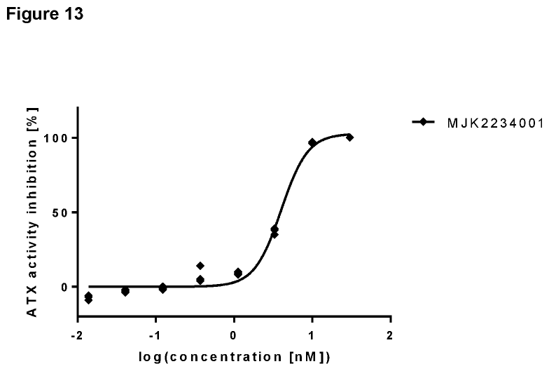

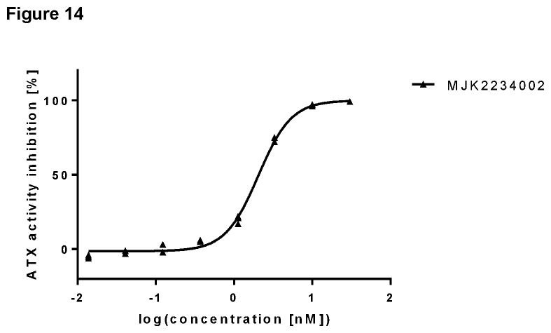

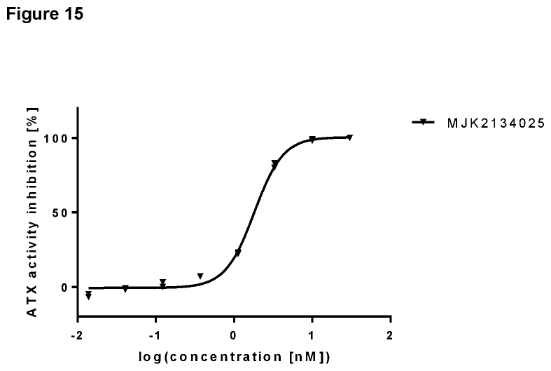

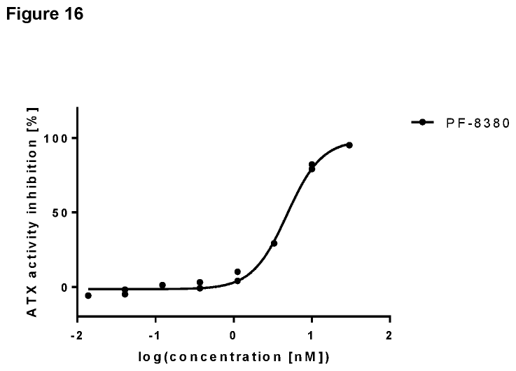

Compounds MJK2234002 and MJK2134025 showed almost equal ATX-inhibitory activity (see also FIGS. 13-17). The autotaxin reaction was performed in three parallel replicates for the target inhibitors MJK2234001 and MJK2234002 and two replicates for MJK2134025 and two replicates for the positive control BrP-LPA. All ATX-tests were performed using the protocol as described above with minor modifications. Those modifications refer to the dilution principle:

### 2.4 Preparation of a Dilution Series of the Inhibitor

A solution of 10 mM of the inhibitor to test in DMSO was received. Adding 40.5 μL of this solution to 959.5 μL DMSO gave a concentration of 405 μM. Adding 820 μL water to 180 μL of the previous solution yielded a total volume of 960 μL (volume reduction caused by solving DMSO in water) and an inhibitor concentration of 75.94 μM. Adding additional 40 μL water yielded a total volume of 1000 μL and an inhibitor concentration of 72.9 μM. For a threefold dilution series 800 μL of 18% DMSO in water were dispensed in twelve 1.5 mL test tubes. 400 μL of the 75.9 μM inhibitor solution were added to the first vial and the solution was vortexed. For every dilution step 400 μL of the corresponding previous dilution step were transferred and mixed by vortexing. The obtained inhibitor concentrations were 72.9 μM, 24.3 μM, 8.10 μM, 2.70 μM, 900 nM, 300 nM, 100 nM, 33.3 nM, 11.1 nM, 3.70 nM, 1.23 nM, 0.41 nM and 0.14 nM. The eight concentration steps from 300 nM to 0.14 nM were used in the assay. During the assay, the final concentration of the inhibitor during the reaction was 1/10th of the added inhibitor solution.

### Example 3

### 3.1 Microsomal Stability Assay

A microsomal stability assay was performed according to an internal standard procedure. Positive control compound diclofenac and three test items were freshly prepared as sub-dilutions in phosphate buffer (100 mM, pH 7.4) at concentrations of 200 μM and 20 μM, respectively. All incubations were conducted in triplicate in glass screw neck vials (1.5 mL, flat bottom, Macherey-Nagel, Düren, Germany) on a Thermomixer C (Eppendorf, Germany) at 37° C. and 1500 rpm. The incubation mixtures contained 270 μL human liver microsomes (HLM, 1.1 mg·mL⁻¹, UltraPool HLM 150, Corning B.V. Life Science, Amsterdam, the Netherlands), 270 μL NADP regeneration mix (1 mM NADP, 5 mM glucose-6-phosphate, 5 units·mL⁻¹ glucose-6-phosphate dehydrogenase and 5 mM MgCl₂) and 30 μL phosphate buffer to reach a total volume of 570 μL. Negative controls were performed with heat inactivated (80° C., 30 min) HLM solution. Reactions were initiated by addition of diclofenac or the test item solution (30 μL). Aliquots of 100 μL were taken at 1, 15, 30, 60, and 90 minutes and immediately processed with 100 μL acetonitrile at 4° C. to terminate the reactions, and shaken for 2 minutes at 4° C. with 1600 rpm. The mixed samples were further transferred to 15 mm glass inserts (0.2 mL/6×31 mm, Macherey-Nagel, Düren, Germany) fitted in glass screw neck vials, and centrifuged for 15 min at 4° C. with 4000 rpm. The supernatants were withdrawn and subjected to high resolution mass spectrometry analysis without further treatment.

### 3.2 Analysis of Microsomal Stability Assay

Samples were analyzed using a Vanquish Horizon UHPLC system (Thermo Fisher Scientific, Bremen, Germany) coupled to a Thermo Scientific QExactive HF-X Orbitrap mass spectrometer (Thermo Fisher Scientific, Bremen, Germany) using a UPLC column (ACQUITY HSS T3, Waters, 1.8 μm particle size, 2.1×50 mm dimensions). The column temperature was maintained at 25° C., and the samples were kept at 4° C. The UHPLC system was operated at a flow rate of 0.6 mL/min and an injection volume of 5 μL. Mobile phases consisted of 0.1% (v/v) formic acid in water (Eluent A) and 0.1% (v/v) formic acid in acetonitrile (Eluent B). Chromatographic separation was obtained as follows: 0 to 4 min: a linear gradient from 5 to 98% of Eluent B; 4 to 6.5 min: isocratic at 98% B; 6.5 to 7 min: linear gradient from 98 to 5% B; 7 to 9 min: isocratic at 5% B. The high resolution mass spectra were acquired with electrospray ionization at positive mode. Full scan data were acquired at a resolution of 60,000 full width at half maximum. Ion source parameters were: spray voltage 3.8 kV, capillary temperature 320° C., RF level 40, sheath gas pressure 50, auxiliary gas 10, auxiliary gas heater temperature 300° C. The value for the automatic gain control target was set to 10⁶. A scan range of m/z 150 to 1800 was chosen, and the maximum injection time was set to 200 ms. The chromatographic peak width was set to 15 s.

### 3.3 Calculation of the Results from Microsomal Stability Assay

$$Elimination rate constant ( k ) = ( - gradient )$$

$$Half - life ( {t}_{1 / 2} ) ( min ) = \frac{ln 2}{k}$$

$$V ( μL \cdot {mg}^{- 1} ) = \frac{volume of incubation ( μL )}{protein in the incubation ( mg )}$$

$$Intrinsic clearance in liver microsomes ( {Cl}_{int , micro} ) ( μL \cdot min \cdot {mL}^{- 1} protein ) = \frac{V \times ln 2}{{t}_{1 / 2}}$$

$$Intrinsic clearance ( {Cl}_{int} ) ( mL \cdot {min}^{- 1} \cdot {kg}^{- 1} ) = \frac{ln 2}{{t}_{1 / 2}} \times \frac{V}{1000} \times \frac{mg microsomal protein}{g liver weight} \times \frac{g liver}{kg body weight}$$

$$( 52.5 mg protein {g}^{- 1} liver used for human ; \\ 22. g {kg}^{- 1} liver to body weight used for human )$$

$$Hepatic extraction ratio ( EH ) = \frac{{f}_{u} \times {Cl}_{int}}{Q + {f}_{u} \times {Cl}_{int}}$$

(fᵤ: fraction of drug unbound in plasma, fᵤ=1 used for the calculation; Q: hepatic blood flow, 20.0 mL·min⁻¹·kg⁻¹ for human).

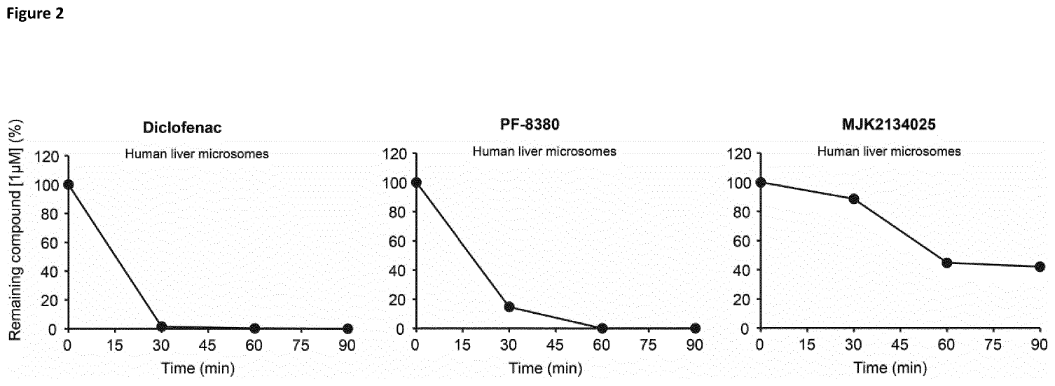

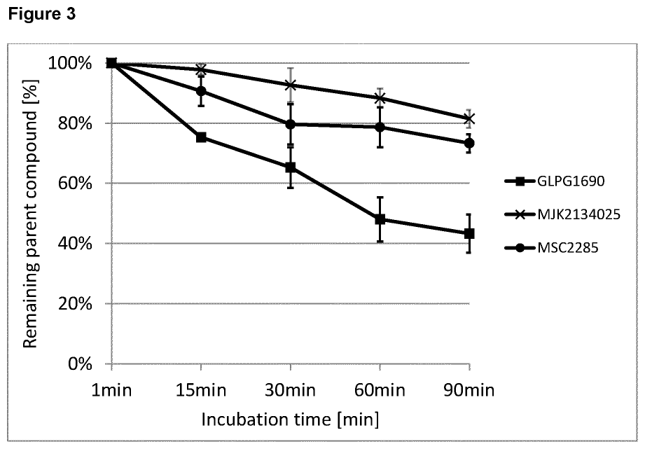

The results have been displayed in FIGS. 2 and 3.

Compound MSC2285 (MSC2285264) corresponds to compound Id of patent application US2012/0202827 A1 and has been prepared for comparison following the synthetic procedure disclosed therein.

### 3.4 Microsomal Stability

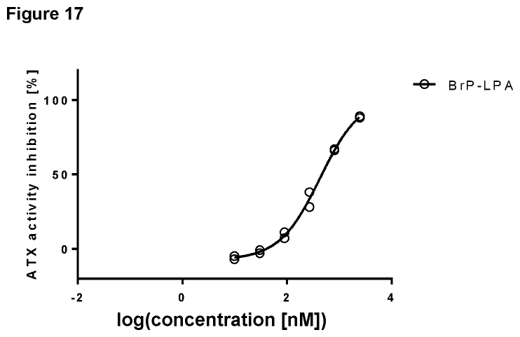

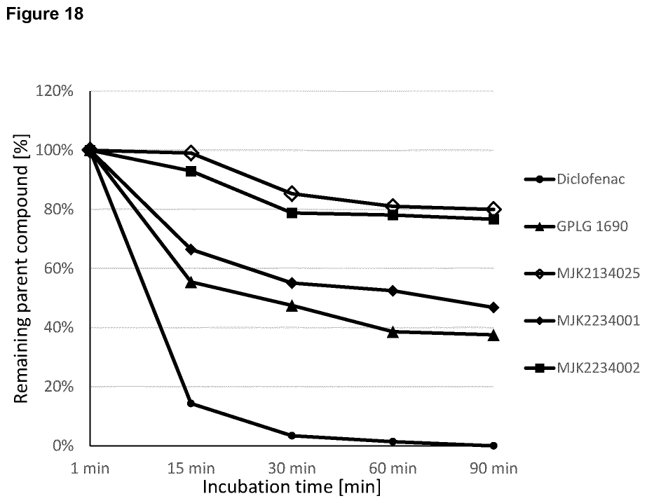

Microsomal stability was determined in analogy to the method as described above, but as single determinations (FIG. 18). The thioether MJK2234001 is metabolized at a comparable speed as the experimental drug GLPG 1690 (ziritaxestat), while MJK2134025 and MJK2234002 are almost equally metabolically stable and superior to all other compounds tested.

### Example 4

### 4.1. Docking Simulations

Simulations were successfully accomplished for all the compounds. Three poses were saved from the docking simulations using each of the different ionization states predicted in the Maestro Suite (by Epik). In addition, the co-crystalized ligand (PF-8380, CAS: 1144035-53-9) was prepared and docked for validation of the workflow. Table 2 shows the corresponding docking scores together with the binding energy calculated using the MM/GBSA method with Prime. The pose considered best for PF-8380 corresponds to the second ionization state, which was supposed to be less likely.

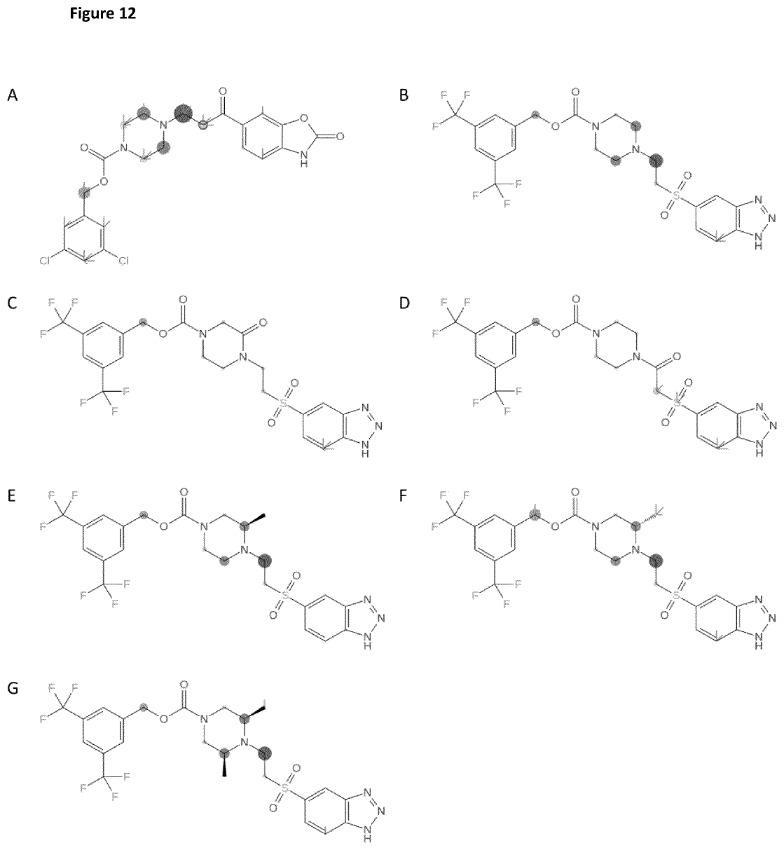

Considering the docking scores, it can be noted that the compounds 1-3 (see FIG. 12 and description for compound structures) have at least one pose with better score than PF-8380.

On the other hand, considering the MM/GBSA binding energy, there are many more poses predicted to have better binding energies than the co-crystalized compound PF-8380, where 1-3 remain as good candidates. Nevertheless, it is impossible to clearly correlate the correct binding mode with the binding energy predicted here. This can be easily observed from one pose of PF-8380, which shows an extremely good binding energy (−70.76kcal/mol) with an unfavorable docking score (−7.38 kcal/mol) and the worst RMSD (4.14 Å).

TABLE 2
Results from docking simulations

| | Ionization | State | Tot | Var- | docking | MMGBSA |
|---|---|---|---|---|---|---|
| Compound | Penalty^{a} | Penalty^{a} | Q | iant | score^{b} | ΔG Bind^{b} |
| MJK2134025 | 0.377 | 0.651 | 0 | 2 | −9.18 | −66.90 |
| MJK2134025 | 0.377 | 0.651 | 0 | 2 | −8.13 | −34.47 |
| MJK2134025 | 0.377 | 0.651 | 0 | 2 | −8.07 | −51.36 |
| MJK2134025 | 0.767 | 0.587 | −1 | 1 | −8.45 | −29.38 |
| MJK2134025 | 0.767 | 0.587 | −1 | 1 | −8.18 | −20.74 |
| MJK2134025 | 0.767 | 0.587 | −1 | 1 | −8.09 | −25.07 |
| 1 | 0.399 | 0.677 | 0 | 2 | −9.99 | −80.00 |
| 1 | 0.399 | 0.677 | 0 | 2 | −9.14 | −72.46 |
| 1 | 0.399 | 0.677 | 0 | 2 | −8.11 | −33.43 |
| 1 | 0.690 | 0.535 | −1 | 1 | −8.50 | −27.90 |
| 1 | 0.690 | 0.535 | −1 | 1 | −7.97 | −26.16 |
| 1 | 0.690 | 0.535 | −1 | 1 | −7.89 | −27.09 |
| 2 | 0.200 | 0.156 | −1 | 1 | −8.12 | −22.88 |
| (MJK2234002) | | | | | | |
| 2 | 0.200 | 0.156 | −1 | 1 | −7.98 | −27.44 |
| (MJK2234002) | | | | | | |
| 2 | 0.200 | 0.156 | −1 | 1 | −7.08 | −25.84 |
| (MJK2234002) | | | | | | |
| 2 | 1.140 | 1.286 | 0 | 2 | −9.91 | −78.97 |
| (MJK2234002) | | | | | | |
| 2 | 1.140 | 1.286 | 0 | 2 | −8.96 | −58.45 |
| (MJK2234002) | | | | | | |
| 2 | 1.140 | 1.286 | 0 | 2 | −7.49 | −29.61 |
| (MJK2234002) | | | | | | |
| 3 | 0.376 | 0.650 | 0 | 2 | −9.52 | −58.42 |
| 3 | 0.376 | 0.650 | 0 | 2 | −8.49 | −34.20 |
| 3 | 0.376 | 0.650 | 0 | 2 | −8.41 | −55.57 |
| 3 | 0.769 | 0.589 | −1 | 1 | −10.09 | −60.85 |
| 3 | 0.769 | 0.589 | −1 | 1 | −8.62 | −19.37 |
| 3 | 0.769 | 0.589 | −1 | 1 | −7.40 | −27.00 |
| 4 | 0.376 | 0.650 | 0 | 2 | −8.04 | −49.00 |
| 4 | 0.376 | 0.650 | 0 | 2 | −7.76 | −35.59 |
| 4 | 0.376 | 0.650 | 0 | 2 | −7.34 | −23.51 |
| 4 | 0.769 | 0.589 | −1 | 1 | −8.53 | −27.29 |
| 4 | 0.769 | 0.589 | −1 | 1 | −8.36 | −25.20 |
| 4 | 0.769 | 0.589 | −1 | 1 | −8.31 | −25.03 |
| 5 | 0.376 | 0.649 | 0 | 2 | −8.70 | −27.17 |
| 5 | 0.376 | 0.649 | 0 | 2 | −8.26 | −50.32 |
| 5 | 0.376 | 0.649 | 0 | 2 | −6.70 | −37.25 |
| 5 | 0.771 | 0.590 | −1 | 1 | −8.82 | −25.95 |
| 5 | 0.771 | 0.590 | −1 | 1 | −8.15 | −29.01 |
| 5 | 0.771 | 0.590 | −1 | 1 | −7.39 | −13.54 |
| PF-8380 | 0.068 | 0.051 | 0 | 1 | −9.85 | −27.03 |
| PF-8380 | 0.068 | 0.051 | 0 | 1 | −9.85 | −43.92 |
| PF-8380 | 0.068 | 0.051 | 0 | 1 | −9.56 | −26.94 |
| PF-8380 | 1.819 | 1.740 | −1 | 2 | −9.85 | −22.22 |
| PF-8380 | 1.819 | 1.740 | −1 | 2 | −8.84 | −22.15 |
| PF-8380 | 1.819 | 1.740 | −1 | 2 | −8.35 | −24.02 |
| PF-8380 | 1.980 | 2.121 | 1 | 3 | −7.66 | −39.43 |
| PF-8380 | 1.980 | 2.121 | 1 | 3 | −7.38 | −70.76 |
| PF-8380 | 1.980 | 2.121 | 1 | 3 | −7.29 | −26.32 |

^{a}Obtained from Epik, implemented in Maestro to define the most favorable species at a specified pH (7 ± 2).
^{b}Docking score and binding energies given in kcal/mol

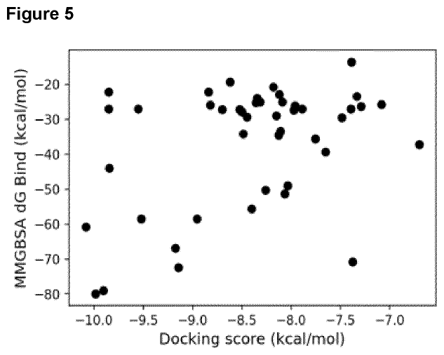

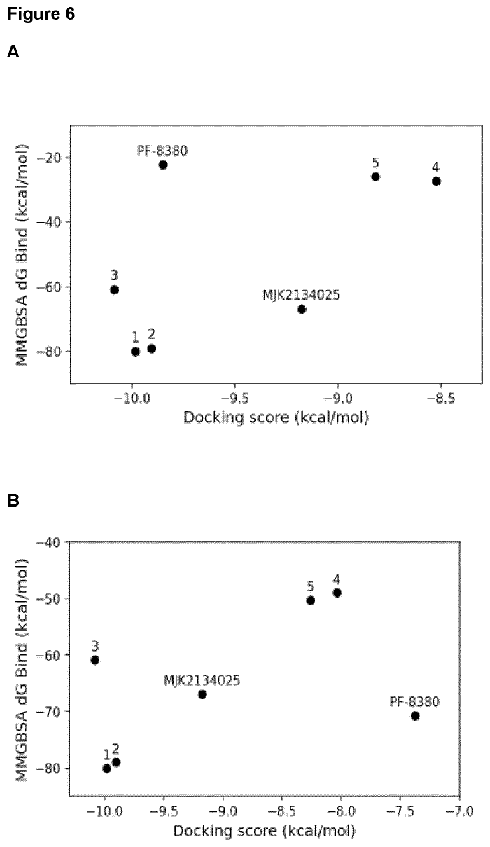

FIG. 5 shows a comparison between docking scores and binding energies. Most obvious differences are reflected by the best pose for each compound (FIG. 6). Regardless of the measurement (docking score or binding energy), the newly suggested compounds are not closely related to the pose of the co-crystalized ligand (PF-8380).

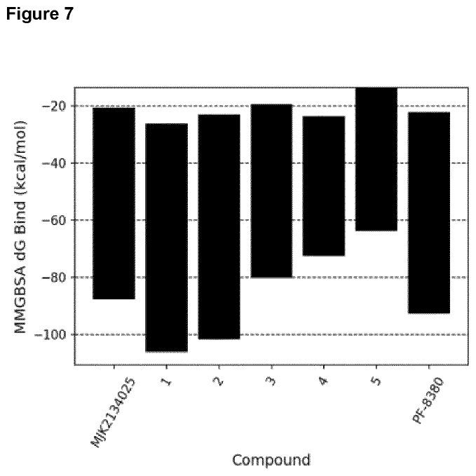

Considering the differences between docking scores and binding energies, FIG. 7 displays the ranges of binding energies found for each compound.

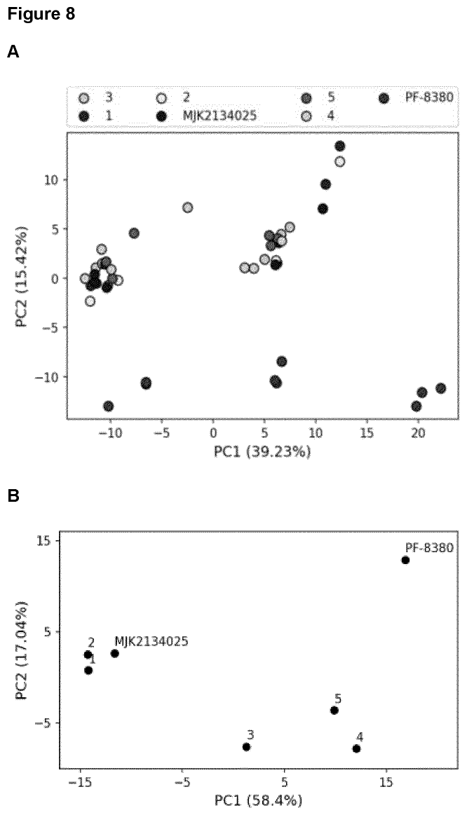

During the docking simulations, the interaction of the ligands with the residues of the binding pocket was calculated. Principal Component Analysis over those interactions (>300 different values per ligand as energy terms decomposed by type of interaction) was carried out (FIG. 8). The generated grouping is mainly due to the different ionization states considered for each compound. The analysis was repeated considering only the best poses according to the docking scores. Recurrent similarities were observed, e.g. 1 and 2 (see Figures and description of Figures for compound numbers) are quite similar in terms of their interactions.

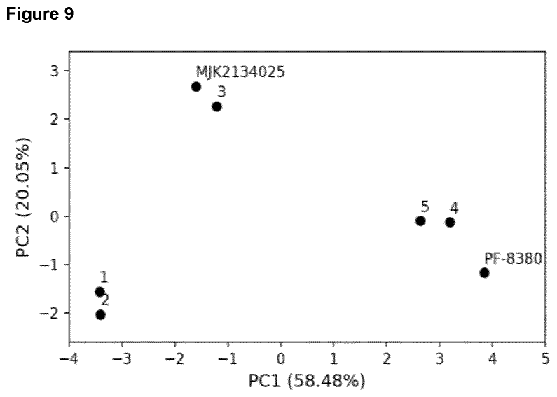

A similar analysis was performed using interaction fingerprints instead (ones and zeros for presence or absence of interaction with certain residues within the target rather than specific energy values). The outcome showed some conserved characteristics as previously found (FIG. 9).

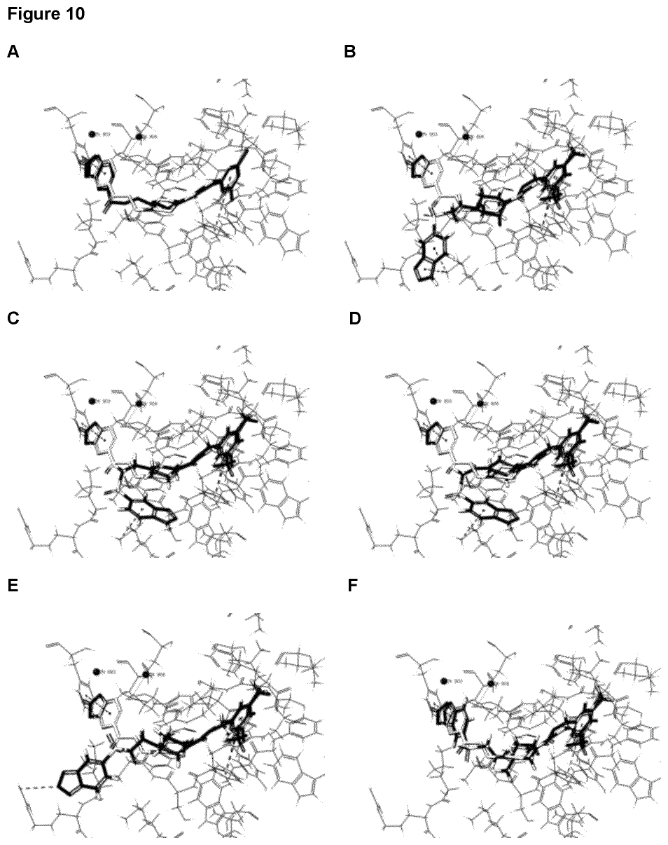

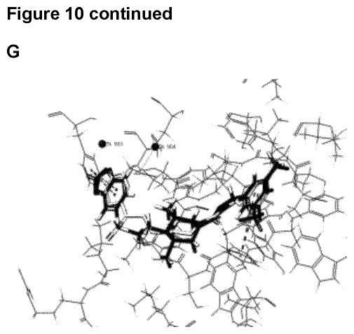

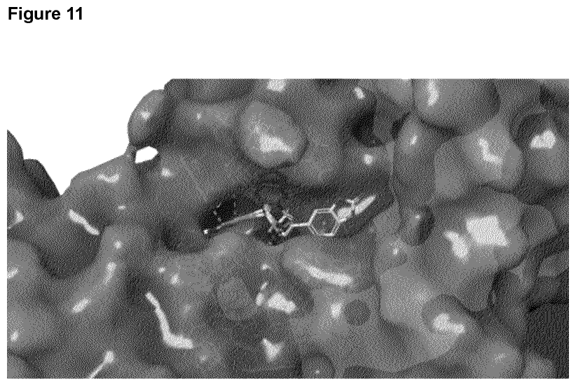

FIG. 10 depicts the structures of the best poses according to the docking scores, always compared to PF-8380. From the visual inspection, it is evident that 4 and 5 present the highest pose similarity with the crystal structure (FIG. 10F, G). They show low docking scores, though. 1 and 2 show a similar conformation (FIG. 10C, D), while 3 is readily distinguishable (FIG. 10E).

### Workflow Used:

1) 3D structure generation from SMILES.
2) Tautomerization/Ionization (based on pKa's) at pH 7±2 (default).
3) Conformational search in 5 kcal/mol window.
4) Geometry optimization using AM1 semi-empirical.
5) Docking simulation (“classical”).
6) Calculation of binding energies (Prime, MM/GBSA)

### 4.2 Simulations of Metabolism

Simulations for site of metabolism (SOM) were carried out using the three CYP available forms within the software (CYP2C9, CYP2D6, and CYP3A4; while the latter is only available for intrinsic reactivity calculations, the other two include Fe accessibility and score from induced-fit docking). According to Fehler! Verweisquelle konnte nicht gefunden werden.2, no reactivity for the N atoms of the piperazine moiety was predicted, which might be a liability of the method. The overall result was identical regardless of the isoform used. Therefore results against CYP2C9 are shown for representation.

As expected, the α-positions to the N of the piperazine (C4, C6, and C8; arbitrary numbering) were predicted as highly reactive in the co-crystalized structure (PF-8380). Similarly, MJK2134025 showed reactivity in the same positions (Table 1). Introduction of methyl groups either on C4 or both C4 and C6 (compounds 3-5, see Figures and description of Figures for compound numbers) did not reduce the suspected metabolization rate (FIG. 1 and Table 1). In contrast, the introduction of carbonyl groups on C4 or C8 (1 and 2, respectively) provided significant reduction in the overall SOM score (FIG. 1), which would indicate that those compounds are less prone to CYP mediated metabolic clearance and give rise to improved pharmacokinetic properties.

### Example 5: Pharmacokinetics and Effects in Mouse Models

### 5.1 Fasting-Induced Hyperphagia Model

C57Bl/6J male animals (at least 10-12 weeks old) were acclimatized in the facility for 7 days prior to the experiment and habituated to the experimental conditions. Animals were matched for weight and/or age. Experiments were performed during the light phase after a fasting period of at least 16-18 h (started 1 h before beginning of the dark phase on the previous day), while water was available ad libitum. For assessment of food consumption, food was weighted before and after an interval of 60 min (FIG. 18).

### 5.2 Dosing

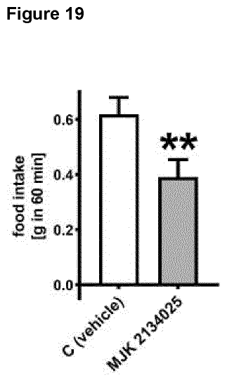

MJK2134025 and GLPG 1690 were each administered as wet milled aqueous micro-suspensions at a concentration of 3 mg/g in 1% carboxymethyl cellulose and 0.5% Tween 80. Dosing of the formulations was performed at 10.0 g/kg body weight, corresponding to 30 mg/kg body weight. The food intake was measured (FIG. 19).

### 5.3 Plasma Sample Collection

Prior blood collection, minicollect K₃ EDTA vials were opened at room temperature to dry the EDTA in order to avoid blood dilution by the fluid content. Following oral gavage of the test formulations tail vein blood samples were taken at 15 min, 30 min, 1 h, 2 h, 4 h and 8 h. Samples were collected in dried minicollect K₃ EDTA plasma vials. The blood samples (20-25 μl) were mixed and immediately cooled to 4° C. Subsequently, the samples were centrifuged at 3000 g for 5-10 min. Supernatants were collected in Eppendorf tubes and frozen and stored at −80° C. until further processing.

### 5.4 Sample Processing and Bioanalytical Determinations

MJK2134025 and GLPG1690 were quantified in mouse plasma samples by LC-MS. Frozen mouse plasma samples were thawed on ice. Samples were then precipitated at a ratio of 1:10 (v:v) with 5 ng mL⁻¹ 17:0 LPA in methanol (as an internal standard) in a 1.5 mL Eppendorf tube with microinsert (clear glass, flat bottom, 0.2 mL, 31×6 mm, LABSOLUTE, Germany). The Eppendorf tubes were mixed for 2 minutes at 4° C. and 1600 rpm in a Thermomixer C (Eppendorf, Germany). The Eppendorf tubes were centrifuged at 4° C. and 16.1 rcf for 2 minutes. The supernatant was transferred to HPLC vials for UHPLC-HRMS measurements as described in the following section. Calibration standards of MJK2134025 and GLPG1690 were prepared by adding the reference substances of MJK2134025 and GLPG1690 to blank samples (mouse plasma without substance). The calibration curve was generated at 7 concentrations and with a weighting of 1/×. The following concentrations were used for the stock solutions: 0.1, 0.5, 1, 2.5, 10, 50 and 200 μg·mL⁻¹ in methanol. To prepare the 200 μg·mL⁻¹ stock solution, 0.04 mL of 1 mg·mL⁻¹ MJK2134025 and 0.04 mL of 1 mg·mL⁻¹ GLPG1690 were mixed with 0.12 mL methanol. These stock solutions (5 μL each) were added to the blank plasma (45 μL). The sample was then mixed for 2 minutes at 4° C. and 1600 rpm in Thermomixer C (Eppendorf, Germany). Subsequent sample processing was the same as for the samples quantified. All quantifications should be considered as preliminary estimates as stable isotope-labeled reference substances were not available and individual matrix effects cannot be excluded.

All processed plasma samples were analyzed with a Vanquish Horizon UHPLC system (Thermo Fisher Scientific, Bremen, Germany) coupled to a Thermo Scientific QExactive HF-X Orbitrap mass spectrometer (Thermo Fisher Scientific, Bremen, Germany) using a UHPLC column (Luna R Omega 1.6 μm C18 100 Å, 100*2.1 mm). A solution of 5 mM ammonium formate in water containing 0.1% formic acid (eluent A) and a 5 mM solution of ammonium formate in acetonitrile: H₂O (95:5, % v:v) containing 0.1% formic acid (eluent B) were used as the mobile phase. A flow rate of 0.3 mL/min was used under the following gradient conditions: 30% eluent B for 1 minute, then linear increase to 98% in 14 minutes, holding 98% of eluent B for 5 minutes, and then decreasing to 30% in 0.5 minutes, followed by isocratic holding for 3.5 minutes. The column temperature was maintained at 25° C. Samples were kept at 4° C., and 8 μL was injected into the UHPLC-MS. The QExactive HF-X Orbitrap was acquired at a resolution of 60000 at full half-width and in both positive and negative modes. The following HESI source parameters were used: capillary voltage of 3.8 kV (negative mode) and 3.5 kV (positive mode), capillary temperature of 320° C., funnel RF level 40, sheath gas pressure 49 (N₂>95%), auxiliary gas 10 (N₂>95%), auxiliary gas heater temperature 300° C. The automatic gain control (AGC) was set to 10E+6. A scan range of m/z 400 to 600 was selected, and the injection time was set to 200 ms. Integration of the peak areas of the compounds of interest was performed using TraceFinder 4.1 SP3.

### 5.5 Pharmacokinetic Evaluation

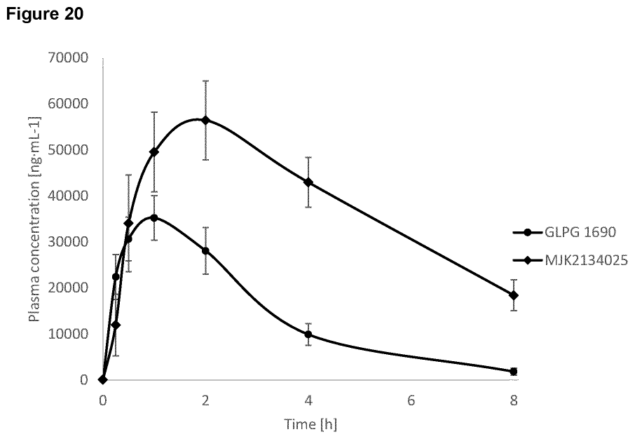

Measured concentrations were listed and summarized for each sampling time point and animal by calculation of means and standard deviation per time point (FIG. 20). The PK assessment was performed based on mean concentrations. The following PK parameters were determined by non-compartmental methods using Phoenix WinNonlin 7.0: Cₘₐₓ, tₘₐₓ, AUCₐₗₗ (Table 4).

TABLE 4
Pharmacokinetic data.

| PK Parameters | GLPG1690 | MJK2134025 |
|---|---|---|
| Cₘₐₓ [ng · mL⁻¹] | 35234.4 | 56426.63 |
| Tₘₐₓ [h] | 1 | 2 |
| AUCₐₗₗ [h ng · mL⁻¹] | 118871.2 | 303182.3 |

### 5.6 CSF Sample Collection

Mice were injected with a mixture of ketamine (100 mg/kg) and xylazine (10 mg/kg) and placed in the stereotaxic frame when deeply anesthetized. The head overlying skin was incised to expose the skull and the posterior neck muscles. The latter were cut off until the cisterna magna was visible through the translucent dura mater. After cleaning any blood residue with a cotton swab, the CSF was collected using a 31-gauge insulin needle (Becton Dickinson) and stored at −80° C. until further processing.

### 5.7 Method for the Relative Analysis of 18:0 LPA in CSF Samples

The methanol extraction of lysophosphatidic acid and MJK2134025 from CSF samples was conducted according to Zhao, Z. and Xu, Y. [8] and Okudaira, M. et al. [9]. Briefly, the frozen CSF sample were thawed on ice. Samples were then precipitated at a ratio of 1:10 (v:v) with 5 ng/ml 17:0 LPA in methanol (as an internal standard) in a 1.5 mL Eppendorf tube with micro insert (clear glass, flat bottom, 0.2 mL, 31×6 mm, LABSOLUTE, Germany). The Eppendorf tubes were mixed for 2 min at 4° C. and 1600 rpm in the Thermomixer C (Eppendorf, Germany). Subsequently, Eppendorf tubes were centrifuged for 2 minutes at 4° C. and 16.1 rcf. The supernatants were transferred to HPLC vials for UHPLC-MS measurements as described in the following section.

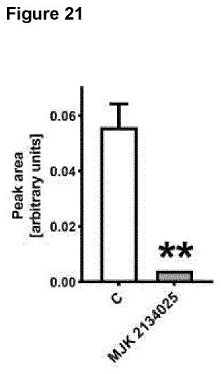

All CSF samples were analyzed using a Vanquish Horizon UHPLC system (Thermo Fisher Scientific, Bremen, Germany) coupled to a Thermo Scientific QExactive HF-X Orbitrap mass spectrometer (Thermo Fisher Scientific, Bremen, Germany) using a UHPLC column (C18 CAPCELL PAK ACR column (1.5×250 mm: Osakasoda, Osaka, Japan). For the mobile phase, a solution of 5 mM ammonium formate in water with 0.1% formic acid (eluent A) and a 5 mM solution ammonium formate in acetonitrile: H₂0 (95:5, % v: v) with 0.1% formic acid (eluent B) was used. A flow rate of 0.5 mL/min was applied under the following gradient conditions: 65% eluent B for 1 min, afterwards linear increase to 95% in 3 min, hold 95% of eluent B for 2 min and then decreased to 65% in 0.5 min, followed by an isocratic hold for 1.5 min. The column temperature was maintained at 25° C. The samples were kept at 4° C. and samples of 15 μL were injected into the UHPLC-MS. The mass spectra were acquired at a resolution of 60000 full width at half maximum and at positive and negative ion switching mode. The following HESI source parameters were applied: spray voltage 3.8 kV (negative mode) and 3.5 kV (positive mode), capillary temperature 320° C., funnel RF level 40, sheath gas pressure 49 (N2>95%), auxiliary gas 10 (N2>95%), auxiliary gas heater temperature 300° C. The automatic gain control (AGC) target was set to 10E+6. A scan range of m/z 400 to 600 was chosen, and the injection time was set to 200 ms. The integration of the peak areas of compounds of interest were done with TraceFinder 4.1 SP3. Due to very limited CSF sample volumes and numbers, a preliminary determination of LPA 18:0 (FIG. 21) was performed relative based on peak area ratios.

### 6. Cytotoxicity Testing

Hela cells (DSM ACC 57) were grown in RPMI 1640 medium supplemented with 10 mL·L⁻¹ ultraglutamine 1 (CAMBREX 17-605E/U1), 550 μL·L⁻¹ gentamicin sulfate (50 mg·mL⁻¹, CAMBREX 17-518Z) and 10% heat inactivated fetal bovine serum (GIBCO Life Technologies 10270-106) at 37° C. in a 5% CO₂ atmosphere in high density polyethylene flasks (NUNC 156340). Cells were pre-incubated for 48 hours in the absence of test substances. Subsequently, Hela cells were incubated with serial dilutions of the test substances in 96 well microplates for 72 hours at 37° C. in a humidified atmosphere and 5% CO₂. After incubation, the cytolytic effect of compounds was analyzed relative to the negative control (DMSO) using a colorimetric assay (methylene blue). The adherent Hela cells were fixed by glutaraldehyde (MERCK 1.04239.0250) and stained with a 0.05% solution of methylene blue (SERVA 29198) for 15 min. After gentle rinsing, the stain was eluted through addition of 0.2 mL hydrochloric acid (0.33 M) to each well. The absorptions were measured at 660 nm in a SUNRISE microplate reader (TECAN). Four replicates were assayed for each substance. The half-cytotoxic concentration (CC₅₀) was defined as the test compound concentration required for 50% reduction of the viable cell count in the monolayer relative to the respective untreated control. All calculations of CC₅₀ values were performed with the software Magellan (TECAN).

### REFERENCES

1. Trimbuch T et al., (2009) Cell 138: 1222-1235

2. Harrison PJ, Weinberger DR (2005) Molecular psychiatry 10: 40-68; image 5

3. Moolenaar WH, Perrakis A (2011) Nature reviews Molecular cell biology 12: 674-679

4. Javitt DC et al., (2008) Nat Rev Drug Discov 7: 68-83

5. Davis M (ed) (1984) The mammalian startle response. New York, NY: Plenum Press

6. Swerdlow NR et al., (1994) Arch Gen Psychiatry 51: 139-154

7. Braff et al., (2001) Psychopharmacology 156: 234-58

8. Zhao, Z., and Y. Xu. An extremely simple method for extraction of lysophospholipids and phospholipids from blood samples. J Lipid Res. 2010. 51: 652-659.

9. Okudaira, M., A. Inoue, A. Shuto, K. Nakanaga, K. Kano, K. Makide, D. Saigusa, Y. Tomioka, and J. Aoki. Separation and quantification of 2-acyl-1-lysophospholipids and 1-acyl-2-lysophospholipids in biological samples by LC-MS/MS. J. Lipid Res. 2014. 55: 2178-2192.

1. A compound according to general structure (I) 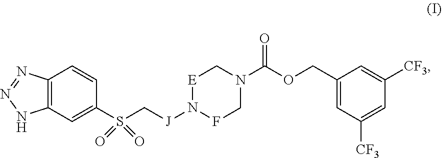
wherein
E is CH₂, C—O or CH(C₁-C₅)alkyl; preferably CH₂;
F is CH₂, C═O or CH(C₁-C₅)alkyl; preferably CH₂;
J is CH₂, or C═O, preferably CH₂;
or a pharmaceutically acceptable carrier, solvate, enantiomer or hydrate thereof.

2. The compound of claim 1, wherein the compound is selected from the group consisting of

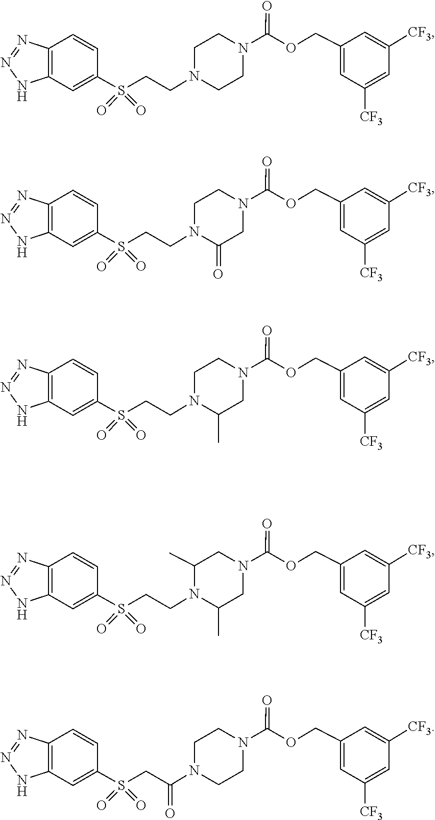 [SMILES: O=C(OCc1cc(C(F)(F)F)cc(C(F)(F)F)c1)N1CCN(CCS(=O)(=O)c2ccc3nn[nH]c3c2)CC1 ; O=C1CN(C(=O)OCc2cc(C(F)(F)F)cc(C(F)(F)F)c2)CCN1CCS(=O)(=O)c1ccc2nn[nH]c2c1 ; CC1CN(C(=O)OCc2cc(C(F)(F)F)cc(C(F)(F)F)c2)CCN1CCS(=O)(=O)c1ccc2nn[nH]c2c1 ; CC1CN(C(=O)OCc2cc(C(F)(F)F)cc(C(F)(F)F)c2)CC(C)N1CCS(=O)(=O)c1ccc2nn[nH]c2c1 ; O=C(CS(=O)(=O)c1ccc2nn[nH]c2c1)N1CCN(C(=O)OCc2cc(C(F)(F)F)cc(C(F)(F)F)c2)CC1]

3. A pharmaceutical composition comprising the compound of claims 1 and 2 and at least one pharmaceutically acceptable excipient.

4. The compound of claim 1 or 2 or the pharmaceutical composition of claim 3 for use in medicine.

5. The compound of claim 1 or 2 or the pharmaceutical composition of claim 3 for use in the prevention or in the treatment of diseases in a subject, in which the inhibition, regulation and/or modulation of autotaxin plays a role, preferably comprising reduction of the level of lysophosphatidic acid (LPA) in the targeted tissue of said subject, more preferably in the brain of said subject.

6. The compound of claim 1, 2 or the pharmaceutical composition of claim 3 for use in the prevention or in the treatment of a central nervous system disorder in a subject, comprising reduction of the level of lysophosphatidic acid (LPA) in the brain of said subject, a fibrotic disease or in the prevention or treatment of cancer.

7. The compound for use or the pharmaceutical composition of claim 6 for use, wherein
a) the central nervous system disorder is a psychiatric disorder and/or
b) the fibrotic disease is selected from thre group consisting of idiopathic lung fibrosis and liver fibrosis

8. The compound for use of claim 6, wherein the central nervous system disorder is a neurological disorder.

9. The compound for use of claim 7, wherein the psychiatric disorder is selected from the group consisting of schizophrenia, depression, anxiety disorders, susceptibility to stress and stress-related disorders, panic disorders, bipolar disorder, obesity, eating disorders and ADHD

10. The compound for use of claim 9, wherein the eating disorder is binge-eating disorder.

11. The compound for use of claim 7 or 9, wherein the psychiatric disorder is obesity or an eating disorder leading to obesity.

12. The compound for use of claim 8, wherein the neurological disorder is selected from the group consisting of multiple sclerosis, epilepsy, Alzheimer's disease and ischemic stroke.

13. The compound for use of claim 6, wherein the cancer is selected from fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiosarcoma, rhabdomyosarcoma, colon, carcinoma, pancreatic cancer, breast cancer, ovarian cancer, prostate cancer, squamous cell carcinoma, basal cell carcinoma, adenocarcinoma, syringe-carcinoma, sebaceous gland carcinoma, papillary carcinoma, papillary adenocarcinomas, cystadenocarcinomas, bone marrow carcinoma, bronchogenic carcinoma, renal cell carcinoma, hepatoma, bile duct carcinoma, choriocarcinoma, seminoma, embryonic carcinoma, Wilm's tumour, cervical cancer, testicular tumour, lung carcinoma, small-cell lung carcinoma, bladder carcinoma, epithelial carcinoma, glioma, astrocytoma, medulloblastoma, craniopharyngioma, ependyoma, pinealoma, haemangioblastoma, acoustic neuroma, oligodendroglioma, meningioma, melanoma neuroblastoma, retinoblastoma, leukaemia, lymphoma, multiple myeloma, Waldenstrom's macroglobulinaemia and heavy chain disease.

14. A process, preferably for the preparation of compound (III), comprising
a) the step of converting compound (II) into compound (III) 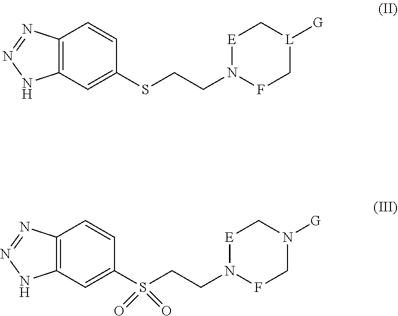
wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1;
optionally, aryl may be substituted with one or more substituents selected from the group consisting of —CF₃, halogen, —OCF₃, —SCF₃, and (C₁-C₆)alkyl.

15. The process of claim 14 wherein in step a), converting compound (II) into (III)
i) is carried out by application of an oxidation-agent in an oxidation step and/or
ii) is carried our by application of an oxidation step comprising application of at least one agent selected from the group consisting of (NH₄)₆Mo₇O₂₄·4H₂O, H₂O₂, and/or mCPBA, preferably mCPBA and/or
iii) comprises formation of intermediate compound (IV) 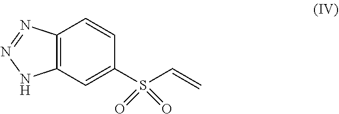
and/or
iv) addition of compound (V) at the vinyl group of compound (IV). 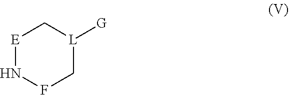
wherein
E is CH₂, C═O or CH(C₁-C₅)alkyl; preferably CH₂;
F is CH₂, C═O or CH(C₁-C₅)alkyl; preferably CH₂;
G is H, —C(O)O(C₁-C₆)alkyl, or —C(O)O(CH₂)ₙ(C₅-C₆)aryl; preferably —C(O)O(CH₂)ₙ(C₅-C₆)aryl;
L is N or CH, preferably N or CH and G is H;
n is 1-4, preferably 1-3, more preferably 1.
